(19) United States
(12) Patent Application Publication
(10) Pub. No.: US 2020/0237711 A1
(43) Pub. Date: Jul. 30, 2020

(54) Compositions for Treating and/or Preventing Cancer

(71) Applicant: Boston Biomedical, Inc., Cambridge, MA (US)

(72) Inventors: Chiang Jia Li, Cambridge, MA (US); Eiichiro Ogimura, Osaka (JP); Tetsuya Nakagawa, Osaka (JP); Eiji Sugaru, Osaka (JP); Nobuyuki Koike, Osaka (JP)

(21) Appl. No.: 16/586,049

(22) Filed: Sep. 27, 2019

Related U.S. Application Data
(63) Continuation of application No. PCT/US2018/023827, filed on Mar. 22, 2018.
(60) Provisional application No. 62/478,788, filed on Mar. 30, 2017.

(51) Int. Cl. A61K 31/343; A61K 47/32; A61K 47/38; A61K 47/10; A61K 47/22; A61K 47/26

(52) U.S. Cl. CPC A61K 31/343; A61K 47/32; A61K 47/26; A61K 47/10; A61K 47/22; A61K 47/38

(57) ABSTRACT

The present disclosure provides pharmaceutical compositions, combinations, and uses thereof for treating and/or preventing cancer. For example, a pharmaceutical composition of the present disclosure can also include 2-acetylnaphtho[2,3-b]furan-4,9-dione, a prodrug thereof, a pharmaceutically acceptable salt of any of the foregoing, or a pharmaceutically acceptable solvate of any of the foregoing; and at least one excipient independently being a binder, a disintegrant, a lubricant, a surfactant, one other excipient, or a combination thereof. For example, a combination of the present disclosure can include 2-acetylnaphtho[2,3 b]furan-4,9-dione, a prodrug thereof, a pharmaceutically acceptable salt of any of the foregoing, or a pharmaceutically acceptable solvate of any of the foregoing; and at least one second agent independently being; a metabolic inhibitor, a transporter inhibitor, a NSAID, or a combination thereof.

This application is a continuation of and claims priority under 35 U.S.C. § 120 from PCT Application No. PCT/US2018/023827 filed on Mar. 22, 2018, which claims the benefit of priority to U.S. Provisional Application Ser. No. 62/478,788, filed on Mar. 30, 2017. The entire contents of each of these priority applications are incorporated herein by reference.

### BACKGROUND

According to recent statistics, about 14 million people are newly diagnosed as having cancer and about 8 million people die of cancer annually in the world. Anti-tumor agents, surgical operations, radiotherapy, immunotherapy, and the like are widely used to treat cancer. Of these, anti-tumor agents are used most often. Anti-tumor agents usually act on the metabolism of cancer cells. However, such metabolic processes occur in not only cancer cells, but also normal cells. As a result, many anti-tumor agents cause unintended side effects.

Recent studies have discovered the presence of cancer stem cells (CSC, cells that are capable of self-renewal). CSCs were reported to closely relate to malignant alteration of cancer. In almost all of human major cancer types including breast cancer, colon cancer, lung cancer, hematological malignancy, and the like, CSCs have been identified. CSCs and bulk cancer cells (cells that occupy the large part of the tumor mass) are significantly different from each other in their biological properties. It has been shown that a CSC is important in the continuous proliferation of a malignant tumor, the metastasis and recurrence of cancer, and the tolerance against anti-tumor agents. Although therapeutic methods targeting bulk cancer cells reduce the tumor size, unless the CSCs are also targeted, a meaningful survival cannot be expected. A compound capable of suppressing CSC (as well as bulk cancer cells) would be useful as a novel anti-tumor agent. In some embodiments, such compound is referred to as a cancer stemness inhibitor.

### SUMMARY OF THE INVENTION

The present disclosure relates to pharmaceutical compositions, combinations, and uses thereof for treating and/or preventing cancer.

One aspect of the present disclosure provides a pharmaceutical composition. In some embodiments, the pharmaceutical composition includes a Compound of the present disclosure. In some embodiments, the pharmaceutical composition includes at least one excipient. In some embodiments, the at least one excipient is at least one binder. In some embodiments, the at least one excipient is at least one disintegrant. In some embodiments, the at least one excipient is at least one other excipient. In some embodiments, the at least one excipient is at least one lubricant. In some embodiments, the at least one excipient is at least one surfactant.

In some embodiments, the pharmaceutical composition includes the Compound in an amount of about 16.7 wt-%; Kollidon VA 64 (copovidone/vinylpyrrolidone-vinyl acetate copolymer) in an amount of about 16.7 wt-%; croscarmellose sodium in an amount of about 16.7 wt-%; mannitol in an amount of about 41.67 wt-%; and Vitamin E TPGS (D-α-tocopheryl polyethylene glycol 1000 succinate) in an amount of about 8.33 wt-%.

In some embodiments, the pharmaceutical composition includes the Compound in an amount of about 80 mg; Kollidon VA 64 (copovidone/vinylpyrrolidone-vinyl acetate copolymer) in an amount of about 80 mg; croscarmellose sodium in an amount of about 80 mg; mannitol in an amount of about 200 mg; and Vitamin E TPGS (D-α-tocopheryl polyethylene glycol 1000 succinate) in an amount of about 40 mg.

In some embodiments, the pharmaceutical composition includes the Compound in an amount of about 16.7 wt-%; Kollidon VA 64 (copovidone/vinylpyrrolidone-vinyl acetate copolymer) in an amount of about 16.7 wt-%; croscarmellose sodium in an amount of about 33.33 wt-%; mannitol in an amount of about 16.7 wt-%; and Vitamin E TPGS (D-α-tocopheryl polyethylene glycol 1000 succinate) in an amount of about 16.7 wt-%.

In some embodiments, the pharmaceutical composition includes the Compound in an amount of about 80 mg; croscarmellose sodium in an amount of about 160 mg; mannitol in an amount of about 80 mg; and Vitamin E TPGS (D-α-tocopheryl polyethylene glycol 1000 succinate) in an amount of about 80 mg.

In some embodiments, the pharmaceutical composition includes the Compound in an amount of about 50.0 wt-%; partially hydrolyzed polyvinyl alcohol in an amount of about 3.0 wt-%; low substituted hydroxypropylcellulose in an amount of about 15.0 wt-%; microcrystalline cellulose in an amount of about 31.0 wt-%; and magnesium stearate in an amount of about 1.0 wt-%.

In some embodiments, the pharmaceutical composition includes the Compound in an amount of about 80.0 mg; partially hydrolyzed polyvinyl alcohol in an amount of about 4.8 mg; low substituted hydroxypropylcellulose in an amount of about 24.0 mg; microcrystalline cellulose in an amount of about 49.6 mg; and magnesium stearate in an amount of about 1.6 mg.

In some embodiments, the pharmaceutical composition includes the Compound in an amount of about 50.0 wt-%; sodium lauryl sulfate in an amount of about 0.5 wt-%; partially hydrolyzed polyvinyl alcohol in an amount of about 2.0 wt-%; low substituted hydroxypropylcellulose in an amount of about 15.0 wt-sodium carboxymethyl starch in an amount of about 4.0 wt-%; microcrystalline cellulose in an amount of about 27.5 wt-%; and magnesium stearate in an amount of about 1.0 wt-%.

In some embodiments, the pharmaceutical composition includes the Compound in an amount of about 80.0 mg; sodium lauryl sulfate in an amount of about 0.8 mg; partially hydrolyzed polyvinyl alcohol in an amount of about 3.2 mg; low substituted hydroxypropylcellulose in an amount of about 24.0 mg; sodium carboxymethyl starch in an amount of about 6.4 mg; microcrystalline cellulose in an amount of about 44.0 mg; and magnesium stearate in an amount of about 1.6 mg.

In some embodiments, the pharmaceutical composition includes a therapeutically effective amount of a Compound of the present disclosure. In some embodiments, the therapeutically effective amount of 2-acetylnaphtho[2,3-b]furan-4,9-dione is a total daily dose ranging from about 10 mg to about 1000 mg. In some embodiments, the total daily dose of 2-acetylnaphtho[2,3-b]furan-4,9-dione is administered in a single time or separately in two or three times. In some embodiments, the total daily dose of 2-acetylnaphtho[2,3-b]furan-4,9-dione is administered separately in two times and each dose is from about 20 mg to about 500 mg. For example, the total daily dose of 2-acetylnaphtho[2,3-b]furan-4,9-dione can be administered separately in two times and each dose is about 80 mg, about 160 mg, about 240 mg, about 320 mg, about 400 mg, about 480 mg, or about 500 mg. In some embodiments, the pharmaceutical composition is administered orally.

Another aspect of the present disclosure provides a combination having a first agent comprising a Compound of the present disclosure; and at least one second agent, each independently selected from metabolic inhibitors and transporter inhibitors. Without being limited to any particular theory or hypothesis, the combination a Compound of the present disclosure and a metabolic inhibitor or a transporter inhibitor can increase the blood concentration and enhance the anti-cancer activity of 2-acetylnaphtho[2,3-b]furan-4,9-dione in animal models.

Specifically, in some embodiments, the present disclosure provides a combination for treating and/or preventing cancer, characterized by combining: (a) a first agent comprising 2-acetylnaphtho[2,3-b]furan-4,9-dione or a pharmaceutically acceptable salt thereof; and (b) at least one second agent comprising at least one type selected from the group consisting of a metabolic inhibitor, a transporter inhibitor, and a combination thereof. In some embodiments, the combination includes (a) a first agent comprising 2-acetylnaphtho[2,3-b]furan-4,9-dione or a pharmaceutically acceptable salt thereof; and (b) at least one second agent each comprising at least one type selected from the group consisting of a metabolic inhibitor, a transporter inhibitor, and a combination thereof, concurrently, separately, or over time.

For example, one of the at least one second agent is a metabolic inhibitor. In some embodiments, the metabolic inhibitor is selected from the group consisting of a reductase inhibitor, an oxidase inhibitor, and a conjugating enzyme inhibitor. In some embodiments, the metabolic inhibitor is a reductase inhibitor. In some embodiments, the reductase inhibitor is selected from the group consisting of an aldo-keto reductase inhibitor (AKR inhibitor), a carbonyl reductase inhibitor (CR inhibitor), an aldehyde reductase inhibitor (ALR inhibitor), and an aldose reductase inhibitor (AR inhibitor). In some embodiments, the reductase inhibitor is an aldo-keto reductase inhibitor (AKR inhibitor). In some embodiments, the reductase inhibitor is a carbonyl reductase inhibitor (CR inhibitor).

In some embodiments, the reductase inhibitor is an aldehyde reductase inhibitor (ALR inhibitor). In some embodiments, the reductase inhibitor is an aldose reductase inhibitor (AR inhibitor). In some embodiments, the aldo-keto reductase inhibitor (AKR inhibitor) is selected from the group consisting of diflunisal, flufenamic acid, mefenamic acid, clobetasol, meclofenamic acid, benzbromarone, ethynylestradiol, clobetasone, dapsone, sulindac, acetohexamide, chlorpromazine, pioglitazone, glibenclamide, losartan, ifenprodil, ketoconazole, salmeterol, megestrol acetate, and glimepiride. In some embodiments, the aldo-keto reductase inhibitor (AKR inhibitor) is selected from the group consisting of diflunisal, flufenamic acid, mefenamic acid, and sulindac.

Another aspect of the present disclosure provides a pharmaceutical composition including a Compound of the present disclosure and optionally a nonsteroidal anti-inflammatory drug (NSAID). In some embodiments, the NSAID is aspirin, sulindac, celecoxib, rofecoxib, valdecoxib, parecoxib, lumiracoxib, etoricoxib, firocoxib, or a combination thereof.

Another aspect of the present disclosure provides uses of a pharmaceutical composition or a combination of the present disclosure for treating or preventing cancer in a subject in need thereof. In some embodiments, the subject is a human.

In some embodiments, the cancer is colorectal cancer, colorectal adenocarcinoma, breast cancer, ovarian cancer, head and neck cancer, skin cancer, melanoma, angiosarcoma, gastric cancer, gastric adenocarcinoma, gastroesophageal adenocarcinoma, lung cancer, pancreatic cancer, prostatic cancer, orchioncus, renal cell carcinoma, hepatocellular carcinoma, cervical cancer, endometrial cancer, urothelial carcinoma, osteosarcoma, Ewing sarcoma, soft tissue sarcoma, brain tumor, multiple myeloma, mesothelioma, leukemia, lymphoma, polycythemia vera, myeloma, esophageal cancer, thyroid carcinoma, biliary tract cancer, chorioepithelioma, infantile malignant solid tumor, or pheochromocytoma. In some embodiments, the cancer is colorectal adenocarcinoma, breast cancer, ovarian cancer, head and neck cancer, melanoma, angiosarcoma, gastric adenocarcinoma, or lung cancer. In some embodiments, the cancer is colorectal adenocarcinoma. In some embodiments, the cancer is ovarian cancer. In some embodiments, the cancer is breast cancer. In some embodiments, the cancer is lung cancer. In some embodiments, the cancer is refractory cancer. In some embodiments, the cancer is recurrent cancer. In some embodiments, the cancer is metastatic cancer. In some embodiments, the cancer is associated with expression of activated STAT3.

For a more complete understanding of the present disclosure and its features and advantages, reference is now made to the following description, taken in conjunction with the accompanying drawings.

### BRIEF DESCRIPTION OF THE DRAWINGS

FIG. 1 illustrates the generally accepted Stat3 pathway in cancer;

FIG. 2 illustrates a comparison of cancer stem cell specific and conventional cancer therapies;

FIG. 3 shows an exemplary comparison of the mouse plasma concentration of 2-acetylnaphtho[2,3-b]furan-4,9-dione (BBI608) after oral administration of DP3_19 and DP3_19v1, two exemplary pharmaceutical compositions of the present disclosure;

FIG. 4 shows an exemplary comparison of the mouse plasma concentration of 2-acetylnaphtho[2,3-b]furan-4,9-dione (BBI608) after oral administration of DP3_19v1, with and without sodium lauryl sulfate, two exemplary pharmaceutical compositions of the present disclosure;

FIG. 5 shows an exemplary comparison of the mouse plasma concentration of 2-acetylnaphtho[2,3-b]furan-4,9-dione (BBI608) after oral administration of DP2A and DP3_19v1, two exemplary pharmaceutical compositions of the present disclosure;

FIG. 6 shows an exemplary comparison of the mouse plasma concentration of 2-acetylnaphtho[2,3-b]furan-4,9-dione (BBI608) after oral administration of DP2A, DP3_19v1, T-45, and T-46, four exemplary pharmaceutical compositions of the present disclosure;

FIG. 7 shows an exemplary production of a metabolic product of 2-acetylnaphtho[2,3-b]furan-4,9-dione in a human liver cytosol fraction according to some embodiments of the present disclosure;

FIG. 8 shows an exemplary inhibition of a metabolic product of 2-acetylnaphtho[2,3-b]furan-4,9-dione in a liver cytosol fraction when a CR inhibitor or AKR inhibitor was added according to some embodiments of the present disclosure;

FIG. 9 shows an exemplary list drugs that have metabolic enzyme inhibitory activity of 2-acetylnaphtho[2,3-b]furan-4,9-dione according to some embodiments of the present disclosure;

FIG. 10 shows the expression of akr genes in a plurality of cancer cells;

FIG. 11 shows an exemplary influence in A549 or H460 cell on the intracellular 2-acetylnaphtho[2,3-b]furan-4,9-dione concentration by addition of a metabolic enzyme inhibitor according to some embodiments of the present disclosure;

FIG. 12 shows an exemplary influence of 2-acetylnaphtho[2,3-b]furan-4,9-dione on cancer cell cytotoxic activity against A549 or H460 cell by addition of a metabolic enzyme inhibitor according to some embodiments of the present disclosure;

FIG. 13 shows an exemplary influence on the 2-acetylnaphtho[2,3-b]furan-4,9-dione concentration in a mouse's plasma or in a tumor mass transplanted to a mouse's abdomen by addition of an agent exhibiting inhibitory activity against a metabolic enzyme according to some embodiments of the present disclosure;

FIG. 14 shows an exemplary influence on 2-acetylnaphtho[2,3-b]furan-4,9-dione concentration in a mouse's plasma by addition of an agent exhibiting inhibitory activity against a metabolic enzyme according to some embodiments of the present disclosure;

FIG. 15 shows an exemplary anti-tumor effect on cancer-bearing mice by addition of an agent exhibiting inhibitory activity against a metabolic enzyme according to some embodiments of the present disclosure.

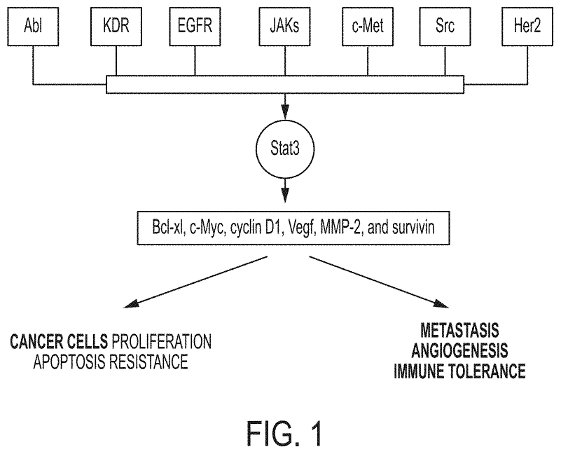

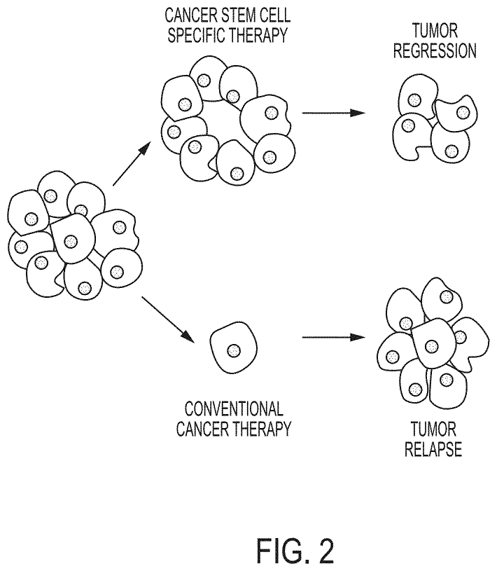

### DETAILED DESCRIPTION

The features and advantages of the present disclosure may be more readily understood by those of ordinary skill in the art upon reading the following detailed description. It is to be appreciated that certain features of the present disclosure that are, for clarity reasons, described above and below in the context of separate embodiments, may also be combined to form a single embodiment and that various features of the present disclosure that are, for brevity reasons, described in the context of a single embodiment, may also be combined so as to form sub-combinations thereof. Embodiments identified herein as exemplary or preferred are intended to be illustrative and not limiting.

Unless specifically stated otherwise, references made in the singular may also include the plural. For example, “a” and “an” may refer to either one or one or more.

When a range of values is listed herein, it is intended to encompass each value and sub-range within that range. For example, “1-5 mg” is intended to encompass 1 mg, 2 mg, 3 mg, 4 mg, 5 mg, 1-2 mg, 1-3 mg, 1-4 mg, 1-5 mg, 2-3 mg, 2-4 mg, 2-5 mg, 3-4 mg, 3-5 mg, and 4-5 mg.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

Certain compounds of the present disclosure may exist in particular geometric or stereoisomeric forms. The present disclosure contemplates all such compounds, including cis and trans-isomers, R- and S-enantiomers, diastereomers, (D)-isomers, (L)-isomers, the racemic mixtures thereof, and other mixtures thereof, as falling within the scope of the present disclosure. Additional asymmetric carbon atoms may be present in a substituent such as an alkyl group. All such isomers, as well as mixtures thereof, are intended to be included in the present disclosure.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C.

Solvates and salts of a compound disclosed herein are also within the scope of the present disclosure. The term “solvate” represents an aggregate that comprises one or more molecules of a compound of the present disclosure with one or more molecules of a solvent or solvents. Solvates of the compounds of the present disclosure include, for example, hydrates.

Examples of the “pharmaceutically acceptable salt” include acid addition salts and base addition salts. Examples of the acid addition salts include inorganic acid salts such as hydrochloride, hydrobromide, sulfate, hydroiodide, nitrate, phosphate, and the like, and organic acid salts such as citrate, oxalate, phthalate, fumarate, maleate, succinate, malate, acetate, formate, propionate, benzoate, trifluoroacetate, methanesulfonate, benzene sulfonate, para-toluene sulfonate, camphor sulfonate, and the like. Examples of the base addition salts include inorganic base salts such as sodium salt, potassium salt, calcium salt, magnesium salt, barium salt, aluminum salt, and the like, and salts of organic bases such as trimethylamine, triethylamine, pyridine, picoline, 2,6-lutidine, ethanolamine, diethanolamine, triethanolamine, tromethamine [tris(hydroxymethyl)methylamine], tert-butylamine, cyclohexylamine, dicyclohexylamine, N,N-dibenzylethylamine, and the like. Further examples include salts of amino acids including basic or acidic amino acids such as arginine, lysin, ornithine, aspartic acid, glutamic acid, and the like.

Prodrugs of a compound disclosed herein are also within the scope of the present disclosure. As used herein, the term “prodrug” refers to a pharmacological derivative of a parent drug molecule or a derivative thereof that requires biotransformation, either spontaneous or enzymatic, within the organism to release the active drug. Unless stated otherwise, prodrugs of a compound of the present disclosure are also contemplated and within the scope of this disclosure. For example, prodrugs are variations or derivatives of compounds disclosed herein and certain derivatives thereof that have groups cleavable under certain metabolic conditions, which when cleaved and optionally further transformed, become the compounds of the present disclosure. Such prodrugs then are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form.

Prodrug forms often offer advantages of solubility, tissue compatibility, or delayed release in the mammalian organism. Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability. As such, those of skill in the art will appreciate that certain of the presently disclosed compounds or derivatives thereof having amino, amido, hydroxy or carboxylic groups can be converted into prodrugs. Prodrugs include compounds or derivatives thereof having an amino acid residue, or a polypeptide chain of two or more (e.g., two, three or four) amino acid residues which are covalently joined through peptide bonds to free amino, or hydroxy or carboxylic acid groups. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. For example, a Compound of the present disclosure can undergo a transformation to produce a phenolic derivative, which can be converted into various prodrugs. When administered, these various prodrugs can be metabolized into the phenolic derivative, which can be further converted to the Compound of the present disclosure. Accordingly, prodrugs prepared from both a compound disclosed herein and its derivatives are within the scope of this disclosure and the appended claims. Prodrugs also include compounds having a carbonate, carbamate, amide or alkyl ester moiety covalently bonded to any of the above substituents disclosed herein.

The compounds, isotopically labelled compounds, salts, solvates, and prodrugs presently disclosed can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, all tautomers are within the scope of the present disclosure.

As used herein, the term “cancer” in a subject refers to the presence of cells possessing characteristics typical of cancer-causing cells, such as uncontrolled proliferation, immortality, metastatic potential, rapid growth and proliferation rate, or/and certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, colon cancer, rectal cancer, cancer of the anal region, stomach cancer, gastric cancer, gastrointestinal cancer, uterine cancer, carcinoma of the fallopian tubes, carcinoma of the endometrium, carcinoma of the vagina, carcinoma of the vulva, Hodgkin's Disease, esophageal cancer, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and both the exemplified cancers and the general terms are included in the term “cancer.” For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

Also included within the term “cancer” is “solid tumor.” As used herein, the term “solid tumor” refers to those conditions, such as cancer, that form an abnormal tumor mass, such as sarcomas, carcinomas, and lymphomas. Examples of solid tumors include, but are not limited to, non-small cell lung cancer (NSCLC), neuroendocrine tumors, thyomas, fibrous tumors, metastatic colorectal cancer (mCRC), and the like. In some embodiments, the solid tumor disease is an adenocarcinoma, squamous cell carcinoma, large cell carcinoma, and the like.

As used herein, “cancer stem cell” (“CSC”) or “cancer stem cells” (“CSCs”) refer to a population of cancer cells that have self-renewal capability and are tumorigenic. They are also called “cancer initiating cells,” “tumor initiating cells,” “cancer stem-like cells,” “stem-like cancer cells,” “aggressive cancer cells,” and “super malignant cancer cells,” etc. The methods of isolating these cells include but are not limited to enrichment by their ability of efflux Hoechst 33342, enrichment of surface markers such as CD133, CD44, and others, and enrichment by their tumorigenic property.

As used herein, the term “cancer stemness inhibitor” refers to a compound that is capable of suppressing CSCs. Without being limited to any particular theory, a cancer stemness inhibitor can target or/and inhibit multiple pathways involved in cancer stem cell's stem-like characteristics. For example, the multiple pathways can involve STAT3, β-CATENIN, NANOG, TCF4, and the like. Cancer stemness inhibitors can be a small molecule or a biologic (including a sugar, a peptide, a protein, a nucleic acid, or a combination thereof). In some embodiments, a cancer stemness inhibitor of the present disclosure is 2-acetylnaphtho[2,3-b]furan-4,9-dione.

As used herein, the term “subject” refers to human and non-human animals, including veterinary subjects. The term “non-human animal” includes all vertebrates, e.g., mammals and non-mammals, such as non-human primates, mice, rabbits, sheep, dogs, cats, horses, cows, chickens, amphibians, and reptiles. In some embodiments, the subject is a human and may be referred to as a patient.

As used herein, the terms “treat,” “treating,” or “treatment” refer, preferably, to an action to obtain a beneficial or desired clinical result including, but not limited to, alleviation or amelioration of one or more signs or symptoms of a disease or condition, diminishing the extent of disease, stabilization (i.e., not worsening) of the state of disease, amelioration or palliation of the disease state, diminishing rate of or time to progression, remission (whether partial or total), whether detectable or undetectable, or/and prevention of a disease or condition. “Treatment” can also mean prolonging survival as compared to expected survival in the absence of treatment. Treatment does not need to be curative and can be an action to administer a compound of the present disclosure to a healthy human who has not developed a disease, for example, to delay or avoid the onset of a disease. Sometimes, this is also referred to as “prevent,” “preventing,” or “prevention.”

As used herein, the term “effective amount” of an active agent refers to an amount sufficient to elicit a desired biological response. As will be appreciated by those of ordinary skill in this art, the effective amount of a compound of the present disclosure may vary depending on such factors as the desired biological endpoint, the pharmacokinetics of the compound, the disease being treated, the mode of administration, or/and the patient.

An “effective amount” of an anti-cancer agent in reference to decreasing cancer cell growth means an amount capable of decreasing, to some extent, the growth of some cancer or tumor cells. The term includes an amount capable of invoking a growth inhibitory, cytostatic and/or cytotoxic effect, and/or apoptosis of the cancer or tumor cells.

A “therapeutically effective amount” in reference to the treatment of cancer, means an amount capable of invoking one or more of the following effects: (1) inhibition, to some extent, of cancer or tumor growth, including slowing down growth or complete growth arrest; (2) reduction in the number of cancer or tumor cells; (3) reduction in tumor size; (4) inhibition (i.e., reduction, slowing down, or complete stopping) of cancer or tumor cell infiltration into peripheral organs; (5) inhibition (i.e., reduction, slowing down, or complete stopping) of metastasis; (6) enhancement of anti-tumor immune response, which may, but is not required to, result in the regression or rejection of the tumor, or/and (7) relief, to some extent, of one or more symptoms associated with the cancer or tumor. The therapeutically effective amount may vary according to factors such as the disease state, age, sex, and weight of the individual and the ability of one or more anti-cancer agents to elicit a desired response in the individual. A “therapeutically effective amount” is also one in which any toxic or detrimental effects are outweighed by the therapeutically beneficial effects.

The term “treating cancer,” “treatment of cancer,” or an equivalent thereof mean to decrease, reduce, or inhibit the replication of cancer cells; decrease, reduce or inhibit the spread (formation of metastases) of cancer; decrease tumor size; decrease the number of tumors (i.e. reduce tumor burden); lessen or reduce the number of cancerous cells in the body; prevent recurrence of cancer after surgical removal or other anti-cancer therapies; or/and ameliorate or alleviate the symptoms of the disease caused by the cancer.

The terms “combination” or “combinatorial,” as used herein, mean the administration of at least two different agents to treat a disorder, condition, or symptom, e.g., a cancer condition. Such combination therapy may involve the administration of one agent before, during, and/or after the administration of a second agent. The compounds, products, and/or pharmaceutical compositions described herein and the second agent can be administered to a subject, preferably a human subject, in the same pharmaceutical composition. Alternatively, the compounds, products, and/or pharmaceutical compositions described herein and the second agent can be administered concurrently, separately, or sequentially to a subject in separate pharmaceutical compositions. The compounds, products, and/or pharmaceutical compositions described herein and the second agent may be administered to a subject by the same or different routes of administration. In some embodiments, a combination of the present disclosure comprises an effective amount of the compounds, products, and/or pharmaceutical compositions described herein and an effective amount of at least one second agent (e.g., prophylactic or therapeutic agent). For example, the at least one second agent can have a different mechanism of action than the compounds, products, and/or pharmaceutical compositions described herein. In some embodiments, a combination of the present disclosure improves the prophylactic or therapeutic effect of the compounds, products, and/or pharmaceutical compositions described herein and of the second agent by functioning together to have an additive or synergistic effect. In some embodiments, a combination of the present disclosure reduces the side effects associated with the second therapy. The administrations of the agents (including a compound or composition of the present disclosure or a second agent) may be separated in time by up to several weeks, but more commonly within 48 hours, and most commonly within 24 hours.

The terms “synergy” and “synergistic” mean that the effect achieved with the compounds used together is greater than the sum of the effects that results from using the compounds separately, i.e., greater than what would be predicted based on the two active ingredients administered separately. A synergistic effect may be attained when the compounds are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anticancer effect denotes an anticancer effect which is greater than the predicted purely additive effects of the individual compounds of the combination.

As used herein, a “metabolic inhibitor” means an agent that inhibits a metabolic enzyme. Examples of the “metabolic inhibitor” include a reductase inhibitor, an oxidase inhibitor, and a conjugating enzyme inhibitor. Examples of the “metabolic inhibitor” include a reductase enzyme inhibitor and an oxidase inhibitor. An example of the “metabolic inhibitor” is a reductase inhibitor. Without being limited to any particular theory, the use of one or more “metabolic inhibitors” and 2-acetylnaphtho[2,3-b]furan-4,9-dione in combination inhibit the metabolism of 2-acetylnaphtho[2,3-b]furan-4,9-dione and consequently enhance the anti-tumor effect of 2-acetylnaphtho[2,3-b]furan-4,9-dione.

As used herein, a “reductase inhibitor” means an agent that inhibits an enzyme that catalyzes a reduction reaction. Examples of the “reductase inhibitor” include an “aldo-keto reductase inhibitor (AKR inhibitor),” a “carbonyl reductase inhibitor (CR inhibitor),” an “aldehyde reductase inhibitor (ALR inhibitor),” and an “aldose reductase inhibitor (AR inhibitor).” Examples of the “reductase inhibitor” include an aldo-keto reductase inhibitor (AKR inhibitor), a carbonyl reductase inhibitor (CR inhibitor), and an aldehyde reductase inhibitor (ALR inhibitor). Examples of the “reductase inhibitor” include an aldo-keto reductase inhibitor (AKR inhibitor) and a carbonyl reductase inhibitor (CR inhibitor). An example of the “reductase inhibitor” is an aldo-keto reductase inhibitor (AKR inhibitor).

Examples of the “aldo-keto reductase inhibitor (AKR inhibitor)” include diflunisal, flufenamic acid, mefenamic acid, meclofenamic acid, sulindac, salmeterol, clobetasol, ethynyl estradiol, clobetasone, progesterone, megestrol acetate, melengestrol acetate, pregnenolone, chlormadinone acetate, halcinonide, mometasone furoate, tibolone, equilin, budesonide, cyproterone acetate, benzbromarone, dapsone, acetohexamide, chlorpromazine, pioglitazone, glibenclamide, losartan, ifenprodil, ketoconazole, or glimepiride.

Specific examples of the “aldo-keto reductase inhibitor (AKR inhibitor)” include diflunisal, benzbromarone, flufenamic acid, mefenamic acid, and sulindac.

Specific examples of the “aldo-keto reductase inhibitor (AKR inhibitor)” include diflunisal, flufenamic acid, mefenamic acid, and sulindac.

An “oxidase inhibitor” means an agent to inhibit a metabolic enzyme that catalyzes an oxidation reaction.

Examples of the “oxidase inhibitor” include a “flavin-containing monooxygenase inhibitor (FMO inhibitor),” an “alcohol dehydrogenase inhibitor (ADH inhibitor),” an “aldehyde dehydrogenase inhibitor (ALDH inhibitor),” and a “monoamine oxidase inhibitor (MAO inhibitor).” Examples of the “oxidase inhibitor” include an “alcohol dehydrogenase inhibitor (ADH inhibitor)” and an “aldehyde dehydrogenase inhibitor (ALDH inhibitor).” An example of the “oxidase inhibitor” is an aldehyde dehydrogenase inhibitor (ALDH inhibitor).

Examples of the “aldehyde dehydrogenase inhibitor (ALDH inhibitor)” include 4-diethylaminobenzaldehyde, benomyl, citral, cyanamide, disulfiram, molinate, pargyline, and daidzin.

A “conjugating enzyme inhibitor” means an agent to inhibit a conjugationally related metabolic enzyme. Examples of the “conjugating enzyme inhibitor” include a “UGP-glucuronosyltransferase inhibitor (UGT inhibitor),” a “sulfotransferase inhibitor (ST inhibitor),” an “amino acid N-acyl transferase inhibitor,” an “acetyltransferase inhibitor (NAT inhibitor),” a “methyltransferase inhibitor,” and “glutathione S-transferase inhibitor (GST inhibitor).” Examples of the “conjugating enzyme inhibitor” include a “UGT inhibitor,” a “ST inhibitor,” and a “GST inhibitor.” Examples of the “conjugating enzyme inhibitor” include a “UGT inhibitor” and a “GST inhibitor.” An example of the “conjugating enzyme inhibitor” is a “GST inhibitor.”

A “transporter inhibitor” means an agent to inhibit a therapeutic agent from being excreted out of a cell. Examples of the “transporter inhibitor” include an ATP-binding cassette transporter (ABC)A1 inhibitor, an ABCA2 inhibitor, an ABCA3 inhibitor, an ABCR inhibitor, an ABCA5 inhibitor, an ABCA6 inhibitor, an ABCA7 inhibitor, an ABCA8 inhibitor, an ABCA9 inhibitor, an ABCA10 inhibitor, an ABCA12 inhibitor, an ABCA13 inhibitor, a multiple drug resistance (MDR)1 inhibitor, a transporter associated with antigen processing (TAP)1 inhibitor, a TAP2 inhibitor, a MDR3 inhibitor, an ABCB5 inhibitor, an ABCB6 inhibitor, an ABC7M-ABC1 inhibitor, an ABCB9 inhibitor, an ABCB10 inhibitor, a bile salt export pump (BSEP) inhibitor, a multidrug resistance-associated protein (MRP)1 inhibitor, a MRP2 inhibitor, a MRP3 inhibitor, a MRP4 inhibitor, a MRP5 inhibitor, a MRP6 inhibitor, a cystic fibrosis transmembrane conductance regulator (CFTR) inhibitor, a sulphonylurea receptors (SUR)1 inhibitor, a SUR2 inhibitor, an ABCC10 inhibitor, an ABCC11 inhibitor, an ABCC12 inhibitor, an ABCC13 inhibitor, an ATP-binding cassette transporter sub-family D (ALD) inhibitor, an ALD2 inhibitor, a peroxisomal membrane protein (PXMP)1 inhibitor, a ribonuclease L (RNASEL) I inhibitor, an ABC50 inhibitor, an ABCF2 inhibitor, an ABCF3 inhibitor, an ABCG1 inhibitor, an ABCG2 inhibitor, an ABCG4 inhibitor, an ABCG5 inhibitor, and an ABCG8 inhibitor. Specific examples of the “transporter inhibitor” include cyclosporin, verapamil, elacridar, gefitinib, and erythromycin.

The term “nonsteroidal anti-inflammatory drugs (NSAIDs),” as used herein, has its generally accepted meaning. It refers to a drug class that conventionally provides analgesic and antipyretic effects. The NSAID herein may include any of a salicylate including aspirin (acetylsalicylic acid), diflunisal, and salsalate, a propionic acid derivative including ibuprofen, dexibuprofen, naproxen, fenoprofen, ketoprofen, dexketoprofen, flurbiprofen, oxaprozin, and loxoprofen, an acetic acid derivative including indomethacin, tolmetin, sulindac, etodolac, ketorolac, diclofenac, and nabumetone, an enolic acid (oxicam) derivative including piroxicam, meloxicam, tenoxicam, droxicam, lornoxicam, and isoxicam, a fenamic acid derivative (fenamates) including efenamic acid, meclofenamic acid, flufenamic acid, and tolfenamic acid, a selective COX-2 inhibitor including celecoxib, rofecoxib, valdecoxib, parecoxib, lumiracoxib, etoricoxib, and firocoxib, a sulphonanilide including nimesulide, and other NSAID including licofelone (acts by inhibiting LOX (lipooxygenase) & COX and hence known as LOX/COX inhibitor) and lysine clonixinate, and a natural NSAID including hyperforin, figwort, and calcitriol (vitamin D).

In one aspect, the present disclosure provides a composition, including a pharmaceutical composition, for treating cancer. In some embodiments, the pharmaceutical composition includes a compound of the present disclosure. In some embodiments, the compound is a cancer stemness inhibitor. In some embodiments, the compound is chosen from 2-acetylnaphtho[2,3-b]furan-4,9-dione, prodrugs thereof, pharmaceutically acceptable salts of any of the foregoing, or solvates of any of the foregoing. In some embodiments, the compound is chosen from compounds having formula I:

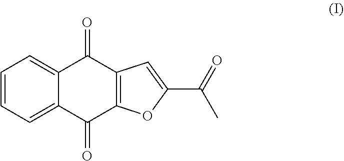 [SMILES: CC(=O)c1cc2c(o1)C(=O)c1ccccc1C2=O]

prodrugs thereof, pharmaceutically acceptable salts of any of the foregoing, and solvates of any of the foregoing. In some embodiments, the compounds having formula I are also referred to as BBI608 or napabucasin. In some embodiments, the compound is chosen from the compounds prepared, for example, by using Examples 8-11 in U.S. Pat. No. 9,084,766, the contents of which are incorporated by reference herein in its entirety. In some embodiments, 2-acetylnaphtho[2,3-b]furan-4,9-dione, the compounds having formula I, the compounds prepared by using Examples 8-11 in U.S. Pat. No. 9,084,766, BBI608, and napabucasin can be used interchangeably.

In some embodiments, the term “Compound of the present disclosure” or “Compound” refers to at least one compound chosen from 2-acetylnaphtho[2,3-b]furan-4,9-dione, compounds having formula I, the compounds prepared by using Examples 8-11 in U.S. Pat. No. 9,084,766, BBI608, or napabucasin, prodrugs thereof, pharmaceutically acceptable salts of any of the foregoing, and solvates of any of the foregoing.

In some embodiments, the amount of a Compound of the present disclosure in a pharmaceutical composition of the present disclosure may range from about 5 wt-% to about 25 wt-%. As used herein, amounts referred to as “wt-%” are weight percentages of the component relative to the total weight of the pharmaceutical composition. In some embodiments, the amount of a Compound of the present disclosure in the pharmaceutical composition may range from about 10 wt-% to about 20 wt-%. In some embodiments, the amount of a Compound of the present disclosure in the pharmaceutical composition is from about 16 wt-% to about 17 wt-%, such as about 16.7 wt-%.

In some embodiments, the amount of a Compound of the present disclosure in the pharmaceutical composition may range from about 20 wt-% to about 80 wt-%. In some embodiments, the amount of a Compound of the present disclosure in the pharmaceutical composition may range from about 40 wt-% to about 60 wt-%. In some embodiments, the amount of a Compound of the present disclosure in the pharmaceutical composition is from about 45 wt-% to about 55 wt-%. In some embodiments, the amount of a Compound of the present disclosure in the pharmaceutical composition is about 50 wt-%.

In some embodiments, a pharmaceutical composition of the present disclosure includes at least one binder. In some embodiments, the pharmaceutical composition includes at least one disintegrant. In some embodiments, the pharmaceutical composition includes at least one other excipient. In some embodiments, a pharmaceutical composition includes at least one component selected from lubricant and surfactant.

In some embodiments, the at least one binder is hydroxypropyl cellulose, alginic acid, carboxymethylcellulose, methylcellulose, copovidone/vinylpyrrolidone-vinyl acetate copolymer, partially hydrolyzed polyvinyl alcohol, or a combination thereof. In some embodiments, the at least one binder is a copovidone/vinylpyrrolidone-vinyl acetate copolymer. In some embodiments, the copovidone/vinylpyrrolidone-vinyl acetate copolymer is Kollidon VA 64 (manufactured by BASF). In some embodiments, the at least one binder is partially hydrolyzed polyvinyl alcohol.

In some embodiments, the amount of the at least one binder in a pharmaceutical composition of the present disclosure is from about 0.5 wt-% to about 5 wt-%. In some embodiments, the amount of the at least one binder in the pharmaceutical composition is from about 1 wt-% to about 4 wt-%. In some embodiments, the amount of the at least one binder in the pharmaceutical composition is from about 1 wt-% to about 3 wt-%. In some embodiments, the amount of the at least one binder in the pharmaceutical composition is from about 1.5 wt % to about 2.5 wt-%. For example, the amount of the at least one binder in the pharmaceutical composition is about 2 wt-%. In some embodiments, the amount of the at least one binder in the pharmaceutical composition is from about 2 wt-% to about 4 wt-%. In some embodiments, the amount of the at least one binder in the pharmaceutical composition is from about 2.5 wt % to about 3.5 wt-%. For example, the amount of the at least one binder in the pharmaceutical composition is about 3 wt-%.

In some embodiments, the amount of the at least one binder in a pharmaceutical composition of the present disclosure is from about 5 wt-% to about 25 wt-%. In some embodiments, the amount of the at least one binder in the pharmaceutical composition is from about 10 wt-% to about 20 wt-%. In some embodiments, the amount of the at least one binder in the pharmaceutical composition is from about 15 wt-% to about 18 wt-%. In some embodiments, the amount of the at least one binder in the pharmaceutical composition is from about 16 wt % to about 17 wt-%. For example, the amount of the at least one binder in the pharmaceutical composition is about 16.7 wt-%.

In some embodiments, the at least one disintegrant is sodium starch glycolate, croscarmellose sodium, crospovidone, sodium carboxymethyl starch, low substituted hydroxypropylcellulose, or a combination thereof. In some embodiments, the at least one disintegrant is croscarmellose sodium. In some embodiments, the at least one disintegrant is low substituted hydroxypropylcellulose. In some embodiments, the at least one disintegrant is sodium carboxymethyl starch.

In some embodiments, the amount of the at least one disintegrant in a pharmaceutical composition of the present disclosure is from about 5 wt-% to about 25 wt-%. In some embodiments, the amount of the at least one disintegrant in the pharmaceutical composition is from about 10 wt-% to about 20 wt-%. In some embodiments, the amount of the at least one disintegrant in the pharmaceutical composition is from about 12 wt-% to about 18 wt-%. In some embodiments, the amount of the at least one disintegrant in the pharmaceutical composition is from about 14 wt % to about 16 wt-%. For example, the amount of the at least one disintegrant in the pharmaceutical composition is about 15 wt-%. In some embodiments, the amount of the at least one disintegrant in the pharmaceutical composition is from about 15 wt-% to about 18 wt-%. In some embodiments, the amount of the at least one disintegrant in the pharmaceutical composition is from about 16 wt % to about 17 wt-%. For example, the amount of the at least one disintegrant in the pharmaceutical composition is about 16.7 wt-%.

In some embodiments, the amount of the at least one disintegrant in the pharmaceutical composition is from about 10 wt-% to about 30 wt-%. In some embodiments, the amount of the at least one disintegrant in the pharmaceutical composition is from about 15 wt-% to about 25 wt-%. In some embodiments, the amount of the at least one disintegrant in the pharmaceutical composition is from about 18 wt % to about 22 wt-%. For example, the amount of the at least one disintegrant in the pharmaceutical composition is about 19 wt-%.

In some embodiments, the amount of the at least one disintegrant in a pharmaceutical composition of the present disclosure is from about 15 wt-% to about 55 wt-%. In some embodiments, the amount of the at least one disintegrant in the pharmaceutical composition is from about 25 wt-% to about 40 wt-%. In some embodiments, the amount of the at least one disintegrant in the pharmaceutical composition is from about 30 wt-% to about 35 wt-%. In some embodiments, the amount of the at least one disintegrant in the pharmaceutical composition is from about 32 wt % to about 34 wt-%. For example, the amount of the at least one disintegrant in the pharmaceutical composition is about 33.33 wt-%.

In some embodiments, the at least one other excipient is mannitol, sorbitol, dibasic calcium phosphate dihydrate, dibasic calcium phosphate anhydrate, tribasic calcium phosphate, microcrystalline cellulose, or a combination thereof. In some embodiments, the at least one other excipient is mannitol. In some embodiments, the at least one other excipient is microcrystalline cellulose.

In some embodiments, the amount of the at least one other excipient in a pharmaceutical composition of the present disclosure is from about 5 wt-% to about 25 wt-%. In some embodiments, the amount of the at least one other excipient in the pharmaceutical composition is from about 10 wt-% to about 20 wt-%. In some embodiments, the amount of the at least one other excipient in the pharmaceutical composition is from about 15 wt-% to about 18 wt-%. In some embodiments, the amount of the at least one other excipient in the pharmaceutical composition is from about 16 wt % to about 17 wt-%. For example, the amount of the at least one other excipient in the pharmaceutical composition is about 16.7 wt-%.

In some embodiments, the amount of the at least one other excipient in a pharmaceutical composition of the present disclosure is from about 15 wt-% to about 45 wt-%. In some embodiments, the amount of the at least one other excipient in the pharmaceutical composition is from about 17.5 wt-% to about 37.5 wt-%. In some embodiments, the amount of the at least one other excipient in the pharmaceutical composition is from about 20 wt-% to about 35 wt-%. In some embodiments, the amount of the at least one other excipient in the pharmaceutical composition is from about 25 wt % to about 30 wt-%. For example, the amount of the at least one other excipient in the pharmaceutical composition is about 27.5 wt-%.

In some embodiments, the amount of the at least one other excipient in the pharmaceutical composition is from about 22 wt-% to about 37 wt-%. In some embodiments, the amount of the at least one other excipient in the pharmaceutical composition is from about 25 wt-% to about 35 wt-%. In some embodiments, the amount of the at least one other excipient in the pharmaceutical composition is from about 30 wt % to about 32 wt-%. For example, the amount of the at least one other excipient in the pharmaceutical composition is about 31 wt-%.

In some embodiments, the amount of the at least one other excipient in a pharmaceutical composition of the present disclosure is from about 20 wt-% to about 60 wt-%. In some embodiments, the amount of the at least one other excipient in the pharmaceutical composition is from about 35 wt-% to about 45 wt-%. In some embodiments, the amount of the at least one other excipient in the pharmaceutical composition is from about 38 wt-% to about 43 wt-%. In some embodiments, the amount of the at least one other excipient in the pharmaceutical composition is from about 41 wt % to about 43 wt-%. For example, the amount of the at least one other excipient in the pharmaceutical composition is about 41.7 wt-%.

In some embodiments, the at least one component is a surfactant. In some embodiments, the surfactant is polyoxyethylene sorbitan alkylate, poloxamer, polyoxyethylene castor oil, polyoxyethylene hydrogenated castor oil, Vitamin E TPGS, or sodium lauryl sulfate. In some embodiments, the polyoxyethylene sorbitan alkylate is polyoxyethylene sorbitan monolaurate or polyoxyethylene sorbitan monooleate. In some embodiments, the polyoxyethylene sorbitan alkylate is polyoxyethylene sorbitan monooleate. In some embodiments, the lipid is a phospholipid, including phosphatidylcholine, phosphatidylserine, phosphatidylethanolamine, phosphatidylglycerol, sphingomyelin, lecithin, or hydrogenated phospholipids; sterol; or cholesterol. In some embodiments, the lipid is a phospholipid including phosphatidylcholine, phosphatidylserine, phosphatidylethanolamine, phosphatidylglycerol, sphingomyelin, lecithin, hydrogenated phospholipids; or cholesterol.

In some embodiments, the at least one surfactant is polysorbates, sodium lauryl sulfate, cyclodextrin, lecithin, Vitamin E TPGS (D-α-tocopheryl polyethylene glycol 1000 succinate), or a combination thereof. In some embodiments, the surfactant is Vitamin E TPGS (D-α-tocopheryl polyethylene glycol 1000 succinate). In some embodiments, the surfactant is sodium lauryl sulfate.

In some embodiments, the amount of the at least one surfactant in a pharmaceutical composition of the present disclosure is from about 0.05 wt-% to about 2 wt-%. In some embodiments, the amount of the at least one surfactant in the pharmaceutical composition is from about 0.1 wt-% to about 2 wt-%. In some embodiments, the amount of the at least one surfactant in the pharmaceutical composition is from about 0.1 wt-% to about 1 wt-%. For example, the amount of the at least one surfactant in the pharmaceutical composition is about 0.5 wt-%.

In some embodiments, the amount of the at least one surfactant in a pharmaceutical composition of the present disclosure is from about 1 wt-% to about 20 wt-%. In some embodiments, the amount of the at least one surfactant in the pharmaceutical composition is from about 5 wt-% to about 12 wt-%. In some embodiments, the amount of the at least one surfactant in the pharmaceutical composition is from about 7 wt-% to about 10 wt-%. In some embodiments, the amount of the at least one surfactant in the pharmaceutical composition is from about 8 wt % to about 9 wt-%. For example, the amount of the at least one surfactant in the pharmaceutical composition is about 8.33 wt-%.

In some embodiments, the amount of the at least one surfactant in a pharmaceutical composition of the present disclosure is from about 5 wt-% to about 25 wt-%. In some embodiments, the amount of the at least one surfactant in the pharmaceutical composition is from about 10 wt-% to about 20 wt-%. In some embodiments, the amount of the at least one surfactant in the pharmaceutical composition is from about 15 wt-% to about 18 wt-%. In some embodiments, the amount of the at least one surfactant in the pharmaceutical composition is from about 16 wt % to about 17 wt-%. For example, the amount of the at least one surfactant in the pharmaceutical composition is about 16.7 wt-%.

In some embodiments, the at least one component is lubricant. In some embodiments, the lubricant is magnesium stearate, talc, paraffin, sodium oleate, sodium lauryl sulfate, magnesium lauryl sulfate, or a combination thereof. In some embodiments, the lubricant is magnesium stearate.

In some embodiments, the amount of the lubricant in a pharmaceutical composition of the present disclosure is from about 0.1 wt-% to about 10 wt-%. In some embodiments, the amount of the lubricant in the pharmaceutical composition is from about 0.1 wt-% to about 5 wt-%. In some embodiments, the amount of the lubricant in the pharmaceutical composition is from about 0.5 wt-% to about 2 wt-%. In some embodiments, the amount of the lubricant in the pharmaceutical composition is from about 0.5 wt % to about 1.5 wt-%. For example, the amount of the lubricant in the pharmaceutical composition is about 1 wt-%.

In some embodiments, the pharmaceutical composition further includes one or more additives, each independently being a fluidizing agent, a coating agent, a solubilizing agent, a solution retarder, an absorption a promoter, a thickener, a dispersing agent, a stabilizer, a sweetening agent, a flavoring agent, a pH regulator, an isotonizing agent, a colorant, an emulsifier, a humectant, a releasing agent, an antiseptic, a preservative, or an antioxidant.

In some embodiments, the additive is lactose, sorbitol, mannitol, crystalline cellulose, calcium carbonate, calcium silicate, anhydrous sodium hydrogen phosphate, methyl cellulose, hypromellose, hydroxypropyl cellulose, povidone, polyvinyl alcohol, carboxymethyl cellulose sodium, ethyl cellulose, cellulose acetate, gum arabic, xanthan gum, powdered tragacanth, gelatin, alginic acid, an alginate salt, low-substituted hydroxypropyl cellulose, carboxymethyl cellulose, corn starch, potato starch, tapioca starch, partly pregelatinized starch, carmellose calcium, croscarmellose sodium, crospovidone, sodium starch glycolate, agar, light anhydrous silicic acid, silicon dioxide, talc, magnesium stearate, calcium stearate, sodium stearyl fumarate, titanium oxide, red diiron trioxide, yellow diiron trioxide, black iron oxide, food color, vegetable oils such as cacao butter, arachis oil, cotton seed oil, corn oil, germ oil, safflower oil, sesame oil, olive oil, soybean oil, and the like; glycerin, polyethylene glycol, propylene glycol, ethyl oleate, ethyl laurate, glycerin fatty acid ester, propylene glycol fatty acid ester, polyoxyethylene fatty acid ester, sorbitan fatty acid ester, polyoxylglycerides, fatty acid, water, saline water, ethyl alcohol, isopropyl alcohol, benzyl alcohol, benzyl benzoate, cetyl alcohol, isostearyl alcohol, cyclodextrin, hydroxypropyl-beta-cyclodextrin, silicone, liquid paraffin, phospholipid such as phosphatidylcholine, phosphatidylserine, phosphatidylethanolamine, phosphatidylglycerol, sphingomyelin, lecithin, hydrogenated phospholipids, and the like; sterol, cholesterol, cholesterol sulfate, ceramide, human serum albumin, kaolin, bentonite, aluminum magnesium silicate, zinc oxide, aspartame, saccharin, saccharin sodium, saccharose, acesulfame K, sucralose, neotame, polyoxyethylene sorbitan alkylate, poloxamer, polyoxyethylene castor oil, polyoxyethylene hydrogenated castor oil, Vitamin E TPGS, sodium lauryl sulfate, sodium carbonate, sodium hydrogen carbonate, sodium hydroxide, glycine, citric acid, sodium citrate, sodium chloride, glucose, a paraoxybenzoate ester, benzalkonium chloride, benzethonium chloride, benzoic acid, sodium benzoate, Vitamin E, propyl gallate, ascorbic acid, sodium sulfite, sodium hydrogensulfite, sodium edetate, erythorbic acid, sodium erythorbate, or cysteine. In some embodiments, the additive is polyoxylglycerides, surfactant, lipid, vegetable oil, glycerin-fatty acid ester, propylene glycol-fatty acid ester, fatty acid, propylene glycol, polyethylene glycol, cellulose or cellulose derivatives, pH regulator, isotonizing agent, or antioxidant. In some embodiments, the additive is polyoxylglycerides, surfactant, lipid, vegetable oil, glycerin-fatty acid ester, propylene glycol-fatty acid ester, fatty acid, propylene glycol, polyethylene glycol, or antioxidant. In some embodiments, the additive is polyoxylglycerides, surfactant, lipid, glycerin-fatty acid ester, propylene glycol-fatty acid ester, fatty acid, or antioxidant. A specific example of the additive can be polyoxylglycerides, surfactant, lipid, or an antioxidant.

In some embodiments, the polyoxylglycerides is caprylocaproyl polyoxylglycerides, lauroyl polyoxylglycerides, linoleoyl polyoxylglycerides, oleoyl polyoxylglycerides, or stearoyl polyoxylglycerides. In some embodiments, the polyoxylglycerides is caprylocaproyl polyoxyl-8 glycerides, lauroyl polyoxyl-32 glycerides, lauroyl polyoxyl-6 glycerides, linoleoyl polyoxyl-6 glycerides, oleoyl polyoxyl-6 glycerides, or stearoyl polyoxyl-32 glycerides. In some embodiments, the polyoxylglycerides is lauroyl polyoxyl-32 glycerides or linoleoyl polyoxyl-6 glycerides. In some embodiments, the polyoxylglycerides is lauroyl polyoxyl-32 glycerides.

In some embodiments, the antioxidant is Vitamin E, Vitamin E TPGS, propyl gallate, ascorbic acid, sodium sulfite, sodium hydrogensulfite, sodium edetate, erythorbic acid, sodium erythorbate, or cysteine. In some embodiments, the antioxidant is Vitamin E or Vitamin E TPGS. In some embodiments, the antioxidant is Vitamin E TPGS.

In some embodiments, the pharmaceutical composition includes a Compound of the present disclosure in an amount ranging from about 5 wt-% to about 25 wt-%; Kollidon VA 64 in an amount ranging from about 5 wt-% to about 25 wt-%; croscarmellose sodium in an amount ranging from about 5 wt-% to about 25 wt-%; mannitol in an amount ranging from about 20 wt-% to about 60 wt-%; and Vitamin E TPGS in an amount ranging from about 1 wt-% to about 20 wt-%. In some embodiments, the pharmaceutical composition includes a Compound of the present disclosure in an amount ranging from about 10 wt-% to about 20 wt-%; Kollidon VA 64 in an amount ranging from about 10 wt-% to about 20 wt-%; croscarmellose sodium in an amount ranging from about 10 wt-% to about 20 wt-%; mannitol in an amount ranging from about 35 wt-% to about 45 wt-%; and Vitamin E TPGS in an amount ranging from about 5 wt-% to about 12 wt-%. In some embodiments, the pharmaceutical composition includes a Compound of the present disclosure in an amount of about 16.7 wt-%; Kollidon VA 64 in an amount of about 16.7 wt-%; croscarmellose sodium in an amount of about 16.7 wt-%; mannitol in an amount of about 41.67 wt-%; and Vitamin E TPGS in an amount of about 8.33 wt-%.

In some embodiments, the pharmaceutical composition may include a Compound of the present disclosure in an amount ranging from about 30 mg to about 130 mg; Kollidon VA 64 in an amount ranging from about 30 mg to about 130 mg; croscarmellose sodium in an amount ranging from about 30 mg to about 130 mg; mannitol in an amount ranging from about 100 mg to about 300 mg; and Vitamin E TPGS in an amount ranging from about 5 mg to about 75 mg. In some embodiments, the pharmaceutical composition may include a Compound of the present disclosure in an amount ranging from about 60 mg to about 100 mg; Kollidon VA 64 in an amount ranging from about 60 mg to about 100 mg; croscarmellose sodium in an amount ranging from about 60 mg to about 100 mg; mannitol in an amount ranging from about 150 mg to about 250 mg; and Vitamin E TPGS in an amount ranging from about 20 mg to about 60 mg. In some embodiments, the pharmaceutical composition may include a Compound of the present disclosure in an amount of about 80 mg; Kollidon VA 64 in an amount of about 80 mg; croscarmellose sodium in an amount of about 80 mg; mannitol in an amount of about 200 mg; and Vitamin E TPGS in an amount of about 40 mg.

In some embodiments, the pharmaceutical composition includes a Compound of the present disclosure in an amount ranging from about 5 wt-% to about 25 wt-%; Kollidon VA 64 in an amount ranging from about 5 wt-% to about 25 wt-%; croscarmellose sodium in an amount ranging from about 15 wt-% to about 55 wt-%; mannitol in an amount ranging from about 5 wt-% to about 25 wt-%; and Vitamin E TPGS in an amount ranging from about 5 wt-% to about 25 wt-%. In some embodiments, the pharmaceutical composition includes a Compound of the present disclosure in an amount ranging from about 10 wt-% to about 20 wt-%; Kollidon VA 64 in an amount ranging from about 10 wt-% to about 20 wt-%; croscarmellose sodium in an amount ranging from about 25 wt-% to about 40 wt-%; mannitol in an amount ranging from about 10 wt-% to about 20 wt-%; and Vitamin E TPGS in an amount ranging from about 10 wt-% to about 20 wt-%. In some embodiments, the pharmaceutical composition includes a Compound of the present disclosure in an amount of about 16.7 wt-Kollidon VA 64 in an amount of about 16.7 wt-%; croscarmellose sodium in an amount of about 33.3 wt-%; mannitol in an amount of about 16.7 wt-%; and Vitamin E TPGS in an amount of about 16.7 wt-%.

In some embodiments, the pharmaceutical composition may include a Compound of the present disclosure in an amount ranging from about 30 mg to about 130 mg; Kollidon VA 64 in an amount ranging from about 30 mg to about 130 mg; croscarmellose sodium in an amount ranging from about 50 mg to about 250 mg; mannitol in an amount ranging from about 30 mg to about 130 mg; and Vitamin E TPGS in an amount ranging from about 30 mg to about 130 mg. In some embodiments, the pharmaceutical composition may include a Compound of the present disclosure in an amount ranging from about 60 mg to about 100 mg; Kollidon VA 64 in an amount ranging from about 60 mg to about 100 mg; croscarmellose sodium in an amount ranging from about 120 mg to about 200 mg; mannitol in an amount ranging from about 60 mg to about 100 mg; and Vitamin E TPGS in an amount ranging from about 60 mg to about 100 mg. In some embodiments, the pharmaceutical composition may include a Compound of the present disclosure in an amount of about 80 mg; Kollidon VA 64 in an amount of about 80 mg; croscarmellose sodium in an amount of about 160 mg; mannitol in an amount of about 80 mg; and Vitamin E TPGS in an amount of about 80 mg.

In some embodiments, the pharmaceutical composition includes a Compound of the present disclosure in an amount ranging from about 25 wt-% to about 75 wt-%; partially hydrolyzed polyvinyl alcohol in an amount ranging from about 0.5 wt-% to about 5 wt-%; low substituted hydroxypropylcellulose in an amount ranging from about 5 wt-% to about 25 wt-%; microcrystalline cellulose in an amount ranging from about 15 wt-% to about 45 wt-%; and magnesium stearate in an amount ranging from about 0.1 wt-% to about 2 wt-%. In some embodiments, the pharmaceutical composition includes a Compound of the present disclosure in an amount ranging from about 40 wt-% to about 60 wt-%; partially hydrolyzed polyvinyl alcohol in an amount ranging from about 1 wt-% to about 4 wt-low substituted hydroxypropylcellulose in an amount ranging from about 10 wt-% to about 20 wt-%; microcrystalline cellulose in an amount ranging from about 22 wt-% to about 37 wt-%; and magnesium stearate in an amount ranging from about 0.5 wt-% to about 1 wt-%. In some embodiments, the pharmaceutical composition includes a Compound of the present disclosure in an amount of about 50 wt-%; partially hydrolyzed polyvinyl alcohol in an amount about 3 wt-low substituted hydroxypropylcellulose in an amount of about 15 wt-%; microcrystalline cellulose in an amount of about 31 wt-%; and magnesium stearate in an amount of about 1 wt-%.

In some embodiments, the pharmaceutical composition may include a Compound of the present disclosure in an amount ranging from about 30 mg to about 130 mg; partially hydrolyzed polyvinyl alcohol in an amount ranging from about 3 mg to about 6 mg; low substituted hydroxypropylcellulose in an amount ranging from about 16 mg to about 32 mg; microcrystalline cellulose in an amount ranging from about 35 mg to about 65 mg; and magnesium stearate in an amount ranging from about 0.5 mg to about 2.5 mg.

In some embodiments, the pharmaceutical composition may include a Compound of the present disclosure in an amount ranging from about 50 mg to about 100 mg; partially hydrolyzed polyvinyl alcohol in an amount ranging from about 4 mg to about 5 mg; low substituted hydroxypropylcellulose in an amount ranging from about 20 mg to about 28 mg; microcrystalline cellulose in an amount ranging from about 45 mg to about 55 mg; and magnesium stearate in an amount ranging from about 1 mg to about 2 mg.

In some embodiments, the pharmaceutical composition may include a Compound of the present disclosure in an amount of about 80 mg; partially hydrolyzed polyvinyl alcohol in an amount of about 4.8 mg; low substituted hydroxypropylcellulose in an amount of about 24 mg; microcrystalline cellulose in an amount of about 49.6 mg; and magnesium stearate in an amount about 1.6 mg.

In some embodiments, the pharmaceutical composition may include a Compound of the present disclosure in an amount ranging from about 20 wt-% to about 80 wt-%; sodium lauryl sulfate in an amount ranging from about 0.1 wt-% to about 2 wt-%; partially hydrolyzed polyvinyl alcohol in an amount ranging from about 0.5 wt-% to about 3 wt-%; low substituted hydroxypropylcellulose in an amount ranging from about 5 wt-% to about 25 wt-%; sodium carboxymethyl starch in an amount ranging from about 1 wt-% to about 7 wt-microcrystalline cellulose in an amount ranging from about 15 wt-% to about 40 wt-%; and magnesium stearate in an amount ranging from about 0.1 wt-% to about 2 wt-%.

In some embodiments, the pharmaceutical composition may include a Compound of the present disclosure in an amount ranging from about 40 wt-% to about 60 wt-%; sodium lauryl sulfate in an amount ranging from about 0.35 wt-% to about 0.65 wt-%; partially hydrolyzed polyvinyl alcohol in an amount ranging from about 1 wt-% to about 2 wt-%; low substituted hydroxypropylcellulose in an amount ranging from about 10 wt-% to about 20 wt-%; sodium carboxymethyl starch in an amount ranging from about 3 wt-% to about 5 wt-%; microcrystalline cellulose in an amount ranging from about 22 wt-% to about 32 wt-%; and magnesium stearate in an amount ranging from about 0.5 wt-% to about 1 wt-%.

In some embodiments, the pharmaceutical composition may include a Compound of the present disclosure in an amount of about 50 wt-%; sodium lauryl sulfate in an amount of about 0.5 wt-%; partially hydrolyzed polyvinyl alcohol in an amount of about 2 wt-%; low substituted hydroxypropylcellulose in an amount of about 15 wt-%; sodium carboxymethyl starch in an amount of about 4 wt-%; microcrystalline cellulose in an amount of about 27.5 wt-%; and magnesium stearate in an amount of about 1 wt-%.

In some embodiments, the pharmaceutical composition may include a Compound of the present disclosure in an amount ranging from about 30 mg to about 130 mg; sodium lauryl sulfate in an amount ranging from about 0.1 mg to about 2 mg; partially hydrolyzed polyvinyl alcohol in an amount ranging from about 1 mg to about 5 mg; low substituted hydroxypropylcellulose in an amount ranging from about 10 mg to about 40 mg; sodium carboxymethyl starch in an amount ranging from about 4 mg to about 9 mg; microcrystalline cellulose in an amount ranging from about 30 mg to about 60 mg; and magnesium stearate in an amount ranging from about 0.5 mg to about 2 mg.

In some embodiments, the pharmaceutical composition may include a Compound of the present disclosure in an amount ranging from about 50 mg to about 100 mg; sodium lauryl sulfate in an amount ranging from about 0.5 mg to about 1 mg; partially hydrolyzed polyvinyl alcohol in an amount ranging from about 2 mg to about 4 mg; low substituted hydroxypropylcellulose in an amount ranging from about 20 mg to about 30 mg; sodium carboxymethyl starch in an amount ranging from about 5 mg to about 7 mg; microcrystalline cellulose in an amount ranging from about 40 mg to about 50 mg; and magnesium stearate in an amount ranging from about 1 mg to about 2 mg.

In some embodiments, the pharmaceutical composition may include a Compound of the present disclosure in an amount of about 80 mg; sodium lauryl sulfate in an amount of about 0.8 mg; partially hydrolyzed polyvinyl alcohol in an amount of about 3.2 mg; low substituted hydroxypropylcellulose in an amount of about 24 mg; sodium carboxymethyl starch in an amount of about 6.4 mg; microcrystalline cellulose in an amount of about 44 mg; and magnesium stearate in an amount of about 1.6 mg.

When a pharmaceutical composition of the present disclosure is administered, the amount of a Compound of the present disclosure used varies depending on the mammal being treated (including symptoms, age, and the like) and the particular mode of administration. In some embodiments, the amount of the Compound will generally be the amount sufficient to produce a desired therapeutic effect. In some embodiments, a total daily dose of the Compound ranges from about 10 mg to about 2000 mg. In some embodiments, a total daily dose of the Compound is about 50 mg, about 80 mg, about 100 mg, about 150 mg, about 160 mg, about 200 mg, about 240 mg, about 250 mg, about 300 mg, about 320 mg, about 350 mg, about 400 mg, about 450 mg, about 480 mg, about 500 mg, about 550 mg, about 560 mg, about 600 mg, about 640 mg, about 650 mg, about 700 mg, about 720 mg, about 750 mg, about 800 mg, about 850 mg, about 880 mg, about 900 mg, about 960 mg, or about 1000 mg. In some embodiments, the total daily dose of the Compound is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg. In some embodiments, the total daily dose of the Compound is about 80 mg, about 160 mg, about 240 mg, about 320 mg, about 480 mg, about 560 mg, about 640 mg, about 720 mg, about 880 mg, or about 960 mg. For example, the total daily dose of the Compound can be about 80 mg, 160 mg, 320 mg, 480 mg, or 960 mg. In some embodiments, the total daily dose of the Compound is 480 mg. In some embodiments, the total daily dose of the Compound is 960 mg. In some embodiments, the total daily dose of the Compound is 1000 mg.

When a pharmaceutical composition of the present disclosure is administered separately in two or more times a day, the amount of the Compound used varies depending on symptoms, age, administration method, and the like. For example, the total daily dose of a Compound of the present disclosure is administered separately in two doses, where each dose is from about 20 mg to about 500 mg. In some embodiments, the total daily dose of the Compound is administered separately in two doses, where each dose is about 80 mg, about 160 mg, about 240 mg, about 320 mg, about 400 mg, about 480 mg, or about 500 mg. In some embodiments, the total daily dose of the Compound is administered separately in two doses, where each dose is about 240 mg, about 480 mg, or about 500 mg.

A pharmaceutical composition of the present disclosure can be formulated in an suitable dosage form for and administered through an administration route selected from the group consisting of oral, nasal, local, rectal, vaginal, or parenteral administration, or intravenous (IV), subcutaneous, or intramuscular injection. Examples of the dosage form include, but not limited to, tablet, capsule, powder, granule, solution, suspension, injection, patch, poultice, and the like. The formulation is produced with a pharmaceutically acceptable additive by a known method.

In one aspect, the present disclosure provides a combination. In some embodiments, the combination is for treating cancer. Without being limited to any particular theory, a combination of the present disclosure can enhance the anticancer activity of a Compound of the present disclosure or/and at least one second agent or/and reduce side effects of the Compound or the at least one second agent. Further, synergistic effects can be observed in a combination of the present disclosure. In some embodiments, the combination includes a Compound of the present disclosure and at least one second agent. In some embodiments, the combination includes a composition disclosed herein and at least one second agent.

In some embodiments, the at least one second agent is a metabolic inhibitor. In some embodiments, the at least one second agent is a reductase inhibitor, an oxidase inhibitor, a conjugating enzyme inhibitor, or a combination thereof. In some embodiments, the at least one second agent is an AKR inhibitor, a CR inhibitor, an ALR inhibitor, an AR inhibitor, or a combination thereof.

In some embodiments, the reductase inhibitor is diflunisal, flufenamic acid, mefenamic acid, meclofenamic acid, sulindac, salmeterol, clobetasol, ethynyl estradiol, clobetasone, progesterone, megestrol acetate, melengestrol acetate, pregnenolone, chlormadinone acetate, halcinonide, mometasone furoate, tibolone, equilin, budesonide, cyproterone acetate, benzbromarone, dapsone, acetohexamide, chlorpromazine, pioglitazone, glibenclamide, losartan, ifenprodil, ketoconazole, glimepiride, or a combination thereof. In some embodiments, the reductase inhibitor is diflunisal, flufenamic acid, mefenamic acid, clobetasol, meclofenamic acid, benzbromarone, ethynyl estradiol, clobetasone, dapsone, sulindac, acetohexamide, chlorpromazine, pioglitazone, glibenclamide, losartan, ifenprodil, ketoconazole, salmeterol, glimepiride, or a combination thereof. In some embodiments, the reductase inhibitor is diflunisal, benzbromarone, flufenamic acid, mefenamic acid, meclofenamic acid, ketoconazole, sulindac, or a combination thereof. In some embodiments, the reductase inhibitor is diflunisal, benzbromarone, flufenamic acid, mefenamic acid, sulindac, or a combination thereof. In some embodiments, the reductase inhibitor is diflunisal. In some embodiments, the reductase inhibitor is flufenamic acid. In some embodiments, the reductase inhibitor is mefenamic acid. In some embodiments, the reductase inhibitor is sulindac.

In some embodiments, the oxidase inhibitor is 4-diethylaminobenzaldehyde, benomyl, citral, cyanamide, disulfiram, molinate, pargyline, daidzin, or a combination thereof.

In some embodiments, the at least one second agent is a transporter inhibitor. In some embodiments, the at least one second agent is cyclosporin, verapamil, elacridar, gefitinib, erythromycin, or a combination thereof. In some embodiments, the at least one second agent is cyclosporin, verapamil, gefitinib, or a combination thereof. In some embodiments, the at least one second agent is gefitinib. Without being limited to any particular theory, the use of a transporter inhibitor and a Compound of the present disclosure in combination can inhibit the Compound from being excreted out of a cancer cell, and consequently enhance the anti-tumor effect of the Compound.

In some embodiments, the at least one second agent is a NSAID. In some embodiments, the NSAID is a salicylate, a propionic acid derivative, an acetic acid derivative, an enolic acid (oxicam) derivative, a fenamic acid derivative (fenamates), a selective COX-2 inhibitor, a sulphonanilide, licofelone, lysine clonixinate, a natural NSAID, or a combination thereof. In some embodiments, the NSAID is aspirin (acetylsalicylic acid), diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, nabumetone, piroxicam, meloxicam, tenoxicam, droxicam, lornoxicam, isoxicam, efenamic acid, meclofenamic acid, flufenamic acid, tolfenamic acid, celecoxib, rofecoxib, valdecoxib, parecoxib, lumiracoxib, etoricoxib, firocoxib, nimesulide, licofelone, lysine clonixinate, hyperforin, figwort, calcitriol (vitamin D), or a combination thereof. In some embodiments, the NSAID is aspirin (acetylsalicylic acid), diflunisal, salsalate, indomethacin, tolmetin, sulindac, etodolac, ketorolac, diclofenac, nabumetone, celecoxib, rofecoxib, valdecoxib, parecoxib, lumiracoxib, etoricoxib, firocoxib, licofelone, or a combination thereof. In some embodiments, the NSAID is aspirin, sulindac, celecoxib, rofecoxib, valdecoxib, parecoxib, lumiracoxib, etoricoxib, firocoxib, or a combination thereof. In some embodiments, the NSAID is aspirin. In some embodiments, the NSAID is sulindac. In some embodiments, the NSAID is celecoxib. In some embodiments, the NSAID is rofecoxib. In some embodiments, the NSAID is firocoxib.

In some embodiments, the at least one second agent is a hormone therapeutic agent, a chemotherapeutic agent, an immunotherapeutic agent, or a cell growth factor inhibitor.

In some embodiments, the at least one second agent is a hormone therapeutic agent. In some embodiments, the at least one second agent is fosfestrol, diethylstilbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, tadenan, mepartricin, raloxifene, ormeloxifene, levormeloxifene, antiestrogen (e.g., tamoxifen citrate, toremifene citrate, and the like), a pill formulation, mepitiostane, testololactone, aminoglutethimide, LH-RH derivatives (LH-RH agonist (e.g., goserelin acetate, buserelin, leuprorelin, and the like), LH-RH antagonist), droloxifene, epitiostanol, ethinyl estradiol sulfonate, aromatase inhibitors (e.g., fadrozole hydrochloride, anastrozole, letrozole, exemestane, vorozole, formestane, and the like), antiandrogens (e.g., flutamide, bicalutamide, nilutamide, and the like), adrenocortical hormone-based agents (e.g., dexamethasone, prednisolone, betamethasone, triamcinolone, and the like), androgen synthesis inhibitors (e.g., abiraterone and the like), retinoid and an agent to retard the metabolism of retinoid (e.g., liarozole and the like), or a combination thereof. In some embodiments, the at least one second agent is dexamethasone.

In some embodiments, the at least one second agent is a chemotherapeutic agent. The chemotherapeutic agent can include an alkylating agent, an antimetabolite, an anti-cancer antibiotic, a plant-derived anti-cancer agent, another chemotherapeutic agent, or a combination thereof.

Accordingly, in some embodiments, the at least one second agent is an alkylating agent. In some embodiments, the at least one second agent is nitrogen mustard, nitrogen mustard N-oxide hydrochloride, chlorambucil, cyclophosphamide, ifosfamide, thiotepa, carboquone, improsulfan tosilate, busulfan, nimustine hydrochloride, mitobronitol, melphalan, dacarbazine, ranimustine, estramustine sodium phosphate, triethylene melamine, carmustine, lomustine, streptozocin, pipobroman, ethoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulfan, trophosphamide, zinostatin stimalamer, adozelesin, cystemustine, bizelesin, or a combination thereof. In some embodiments, the at least one second agent is carboplatin, cisplatin, oxaliplatin, or a combination thereof. In some embodiments, the at least one second agent is carboplatin. In some embodiments, the at least one second agent is oxaliplatin.

In some embodiments, the at least one second agent is an antimetabolite. In some embodiments, the at least one second agent is mercaptopurine, 6-mercaptopurine riboside, thioinosine, methotrexate, pemetrexed, enocitabine, cytarabine, cytarabine ocfosphate, ancitabine hydrochloride, 5-FU based agent (e.g., fluorouracil, tegafur, UFT, doxifluridine, carmofur, galocitabine, emitefur, capecitabine, and the like), aminopterin, nelzarabine, leucovorin calcium, Tabloid, butocin, calcium folinate, calcium levofolinate, cladribine, emitefur, fludarabine, gemcitabine, hydroxycarbamide, pentostatin, piritrexim, idoxuridine, mitoguazone, tiazofurin, ambamustine, bendamustine, or a combination thereof. In some embodiments, the at least one second agent is 5-FU based agent (e.g., fluorouracil, tegafur, UFT, doxifluridine, carmofur, galocitabine, emitefur, capecitabine, and the like), leucovorin, gemcitabine, or a combination thereof. In some embodiments, the at least one second agent is 5-FU based agent (e.g., fluorouracil, tegafur, UFT, doxifluridine, carmofur, galocitabine, emitefur, or capecitabine). In some embodiments, the at least one second agent is leucovorin. In some embodiments, the at least one second agent is gemcitabine.

In some embodiments, the at least one second agent is an anti-cancer antibiotic. In some embodiments, the anti-cancer antibiotic includes include actinomycin D, actinomycin C, mitomycin C, chromomycin A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarkomycin, carzinophilin, mitotane, zorubicin hydrochloride, mitoxantrone hydrochloride, idarubicin hydrochloride, or a combination thereof.

In some embodiments, the at least one second agent is a plant-derived anti-cancer agent. In some embodiments, the plant-derived anti-cancer agent is etoposide, etoposide phosphate, vinblastine sulfate, vincristine sulfate, vindesine sulfate, teniposide, paclitaxel, docetaxel, DJ-927, vinorelbine, irinotecan, topotecan, or a combination thereof.

In some embodiments, the at least one second agent is other chemotherapeutic agent. In some embodiments, the other chemotherapeutic agent is sobuzoxane.

In some embodiments, the at least one second agent is immunotherapeutic agent. An immunotherapeutic agent can be a cell, for example, an immune cell. For example, an immune cell, particularly one that is specific to a tumor, can be activated, cultured, and administered to a patient. In some embodiments, that at least one second agent is a natural killer cell, lymphokine-activated killer cell, cytotoxic T-cell, or dendritic cell. An immunotherapeutic agent can be sipuleucel-T (Provenge).

In some embodiments, the at least one second agent is an antibody. For example, the antibody can bind to a cancer antigen, induce antibody-dependent cell-mediated cytotoxicity, activate the complement system, prevent a receptor from interacting with its ligand, or delivers a chemotherapeutic agent.

In some embodiments, the at least one second agent is an agent targeting cytotoxic T-lymphocyte-associated antigen (CTLA, for example, CTLA4), programmed cell death protein (PD, for example, PD-1), T cell membrane protein (TIM, for example, TIM3), adenosine A2a receptor (A2aR), lymphocyte activation gene (LAG, for example, LAG3), killer immunoglobulin receptor (KIR), or the like. For example, it can be a CTLA4 inhibitor, a PD1 inhibitor, a PDL1, a LAG3 inhibitor, a KIR inhibitor, a B7-H3 ligand, a B7-H4 ligand, or a TIM3 inhibitor. In some embodiments, the at least one second agent is AMP-224, Alemtuzumab, Bavituximab, Bevacizumab, BMS-936559, BMS-986016, Brentuximab vedotin, Cetuximab, Gemtuzumab ozogamicin, Ibritumomab tiuxetan, IMP321, Ipilimumab, Lambrolizumab (MK3475), Lirilumab (BMS-986015), MDX-1105, MGA271, MPDL3280A, Nivolumab, Ofatumumab, Panitumumab, Pembrolizumab, Pidilizumab (CT-011), Rituximab, Tositumomab, Trastuzumab, Tremelimumab (MEDI4736), Urelumab, or a combination thereof. An immunotherapeutic agent can also be a cytokine. In some embodiments, the at least one second agent is Ipilimumab, Nivolumab, Pembrolizumab, or a combination thereof.

In some embodiments, the at least one second agent is an interferon (IFN), interleukin, or the like. In some embodiments, the at least one second agent is interferon (IFNα or IFNβ), type 2 (IFNγ), or type III (IFNλ). In some embodiments, the at least one second agent is interleukin-1 (IL-1), interleukin-1α (IL-1α), interleukin-1β (IL-1β), interleukin-2 (IL-2), interleukin-3 (IL-3), interleukin-4 (IL-4), interleukin-6 (IL-6), interleukin-8 (IL-8), interleukin-10 (IL-10), interleukin-11 (IL-11), interleukin-12 (IL-12), interleukin-13 (IL-13), or interleukin-18 (IL-18), or a combination thereof. In some embodiments, the immunotherapeutic agent is picibanil, krestin, schizophyllan, lentinan, ubenimex, interferon, interleukin, macrophage colony-stimulating factor, granulocyte colony stimulating factor, erythropoietin, lymphotoxin, BCG vaccine, Corynebacterium parvum, levamisole, polysaccharide K, procodazole, anti-CTLA4 antibody, PD-1 antibody, or Toll-like Receptors agonist (e.g., TLR7 agonist, TLR8 agonist, TLR9 agonist, and the like).

In some embodiments, the at least one second agent is an inhibitor of a cell growth factor. Commonly, a cell growth factor includes a factor that is a peptide having a molecular weight of 20,000 or less and exhibits an effect at a low concentration by binding with the receptor. Specifically, EGF (epidermal growth factor) or substances having substantially the same activity thereas (e.g., TGF-alpha and the like), insulin or substances having substantially the same activity thereas (e.g., insulin, IGF (insulin-like growth factor)-1, IGF-2, and the like), FGF (fibroblast growth factor) or substances having substantially the same activity thereas (e.g., acidic FGF, basic FGF, KGK (keratinocyte growth factor), FGF-10, and the like), and other cell growth factors (e.g., CSF (colony stimulating factor), EPO (erythropoietin), IL-2 (interleukin-2), NGF (nerve growth factor), PDGF (platelet-derived growth factor), TGF-beta (transforming growth factor beta), HGF (hepatocyte growth factor), VEGF (vascular endothelial growth factor), heregulin, angiopoietin, and the like).

In one aspect, the present disclosure provides methods of treating a subject in need thereof. In another aspect, the present disclosure provides uses of a Compound, a composition, or/and a combination of the present disclosure in the treatment of a subject in need thereof.

In some embodiments, the subject has a cancer. In some embodiments, the cancer is acute leukemia, chronic lymphocytic leukemia, chronic myelogenous leukemia, polycythemia vera, malignant lymphoma, brain tumor, head and neck cancer, esophageal cancer, thyroid cancer, small cell lung cancer, non-small cell lung cancer, breast cancer, gastric cancer, gallbladder/bile duct cancer, hepatoma, pancreatic cancer, colon cancer, rectal cancer, chorioepithelioma, chorioblastoma, choriocarcinoma, endometrial cancer, cervical cancer, urothelial cancer, renal cell carcinoma, orchioncus, Wilms tumor, skin cancer, malignant melanoma, neuroblastoma, osteosarcoma, Ewing sarcoma, soft tissue sarcoma, or a combination thereof.

In some embodiments, the method or the use includes administering a therapeutically effective amount of a combination of the present disclosure. In some embodiments, a method or a use of the present disclosure includes administering a therapeutically effective amount of a Compound of the present disclosure. In some embodiments, the method or the use includes administering a therapeutically effective amount of a pharmaceutical composition of the present disclosure. In some embodiments, the method or the use includes administering a therapeutically effective amount of the at least one second agent, each of which is discussed in detail herein. In some embodiments, the method or the use includes operation, radiotherapy, gene therapy, thermotherapy, cryotherapy, laser burning therapy, or a combination thereof.

An administration of a Compound of the present disclosure and the at least one second agent is not limited to any particular time, interval, or sequence. They may be administered simultaneously or at any time intervals to a subject. In addition, a mixture of a pharmaceutical composition of the present disclosure and the at least one second agent may be formed. The dosage of the at least one second agent can appropriately be selected based on a dose to be clinically used. Moreover, the mixture ratio of a Composition of the present composition and the at least one second agent can appropriately be selected depending on a subject to be administered, administration route, target disease, symptoms, combinations, and the like. For example, when a subject to be administered is human, 0.01 to 100 parts by weight of the at least one second agent may be used per part by weight of a composition of the present disclosure. Further, for the purpose of suppressing its side effects, it can be used in combination with agents (e.g., the at least one second agent) such as an antiemetic, a sleep-inducing agent, an anticonvulsant, and the like.

In another aspect, disclosed herein are methods of inhibiting, reducing, and/or diminishing cancer stem cell survival and/or self-renewal comprising administering a therapeutically effective amount of at least one pharmaceutical composition comprising a Compound of the present disclosure; at least one disintegrant; at least one other excipient; and at least one component chosen from lubricant and surfactant. In some embodiments, the method includes administering a therapeutically effective amount of at least one second agent, which is discussed in detail herein.

In another aspect, also disclosed herein are methods of treating at least one cancer that is refractory to conventional chemotherapies and/or targeted therapies in a subject comprising administering a therapeutically effective amount of at least one pharmaceutical composition comprising a Compound of the present disclosure; at least one disintegrant; at least one other excipient; and at least one component chosen from lubricant and surfactant. In some embodiments, the method includes administering a therapeutically effective amount of at least one second agent, which is discussed in detail herein.

In another aspect, disclosed herein are methods of treating recurrent cancer in a subject that has failed surgery, chemotherapy, or radiation therapy, comprising administering a therapeutically effective amount of at least one pharmaceutical composition comprising a Compound of the present disclosure; at least one disintegrant; at least one other excipient; and at least one component chosen from lubricant and surfactant. In some embodiments, the method includes administering a therapeutically effective amount of at least one second agent, which is discussed in detail herein.

In another aspect, also disclosed herein are methods of treating or preventing cancer metastasis in a subject, comprising administering a therapeutically effective amount of at least one pharmaceutical composition comprising a Compound of the present disclosure; at least one disintegrant; at least one other excipient; and at least one component chosen from lubricant and surfactant. In some embodiments, the method includes administering a therapeutically effective amount of at least one second agent, which is discussed in detail herein.

In some embodiments, the present disclosure provides a method of treating cancer in a subject, comprising administering a therapeutically effective amount of at least one pharmaceutical composition comprising a Compound of the present disclosure; at least one disintegrant; at least one other excipient; and at least one component chosen from lubricant and surfactant. In some embodiments, the method includes administering a therapeutically effective amount of at least one second agent, which is discussed in detail herein. In some embodiments, the cancer may be chosen from gastric and gastroesophageal adenocarcinoma, colorectal adenocarcinoma, breast cancer, ovarian cancer, head and neck cancer, melanoma, and pancreatic cancer. In some embodiments, the cancer is metastatic pancreatic adenocarcinoma.

In some embodiments, the cancer may be refractory. In some embodiments, the cancer may be recurrent. In some embodiments, the cancer may be metastatic. In some embodiments, the cancer may be associated with expression of activated STAT3. In some embodiments, the cancer may be associated with nuclear β-CATENIN overexpression.

Examples, tables, and figures are provided to facilitate a person with ordinary skill in the art to understand this disclosure and appreciate the appended claims. As such, they are not used to limit the scope of the present disclosure and the appended claims. It should be noted that compound names shown in the following reference examples and Examples do not always follow the IUPAC nomenclature. It should be noted that although abbreviations are sometimes used to simplify a description, these abbreviations are defined the same as the above descriptions.

### EXAMPLES

2-Acetylnaphtho[2,3-b]furan-4,9-dione, BBI608, napabucasin, or a compound having formula I may be synthesized, e.g., according to Examples 8-11 in U.S. Pat. No. 9,084,766.

The human SW480 (colon carcinoma), MIA PaCa-2 (pancreatic carcinoma), and MKN45 (gastric adenocarcinoma) human cell lines were purchased from American Type Culture Collection (ATCC; www.atcc.org/en.aspx; Manassas, Va. 20110; tel. 703-365-2700).

### Example 1. Preparation of DP2A of 2-acetylnaphtho[2,3-b]furan-4,9-dione (BBI608)

A small scale DP2A was prepared by weighing 100 mg BBI608 in a glass vial, adding 8 mL of Labrafil M 2125 CS to BBI608, and mixing the mixture by vortex, followed by adding 2 mL of Gelucire 44/14 to the same mixture and mixing by vortex to get a uniform suspension at 10 mg/mL. This DP2A was used to dose for 100 mg/kg dose regimen.

Large-scale DP2A formulations were prepared by using the components in Table 2.

TABLE 2

| | 125 mg capsule | | 80 mg capsule | |
|---|---|---|---|---|
| Component | mg/capsule | wt-% | mg/capsule | wt-% |
| 2-acetylnaphtho[2,3- | 125 | 27.18 | 80 | 27.18 |
| b]furan-4,9-dione | | | | |
| SLS | 1.2 | 0.27 | 0.8 | 0.27 |
| Gelucire 44/14 | 66.8 | 14.51 | 42.7 | 14.51 |
| Labrifil M2125 CS | 267 | 58.04 | 170.9 | 58.04 |
| Capsule size | size 1 | — | size 1 or 2 | — |

| | | |
|---|---|---|
| Total weight | 460 mg | 294.4 mg |

### Example 2. Preparation of DP3_19 of 2-acetylnaphtho[2,3-b]furan-4,9-dione (BBI608)

A small scale DP3_19 formulation was prepared by weighing BBI608 (2-acetylnaphtho[2,3-b]furan-4,9-dione), Croscarmellose Sodium, Kollidon VA 64, and mannitol in a container. The mixture was hand ground with a mortar and pestle. The required amount of Vitamin E TPGS was added to the mixture and the resulting mixture was hand ground to achieve fine formulation mixture using mortar and pestle.

Large-scale DP3_19 formulations were prepared by using the components in Table 3.

TABLE 3

| Component | mg | wt-% |
|---|---|---|
| 2-acetylnaphtho[2,3-b]furan-4,9-dione | 80 | 16.66 |
| Vitamin E TPGS | 80 | 16.66 |
| Croscarmellose sodium | 160 | 33.33 |
| Kollidon VA 64 | 80 | 16.66 |
| Mannitol | 80 | 16.66 |
| Total weight | 480 | — |

### Example 3. Preparation of DP3_19v1 of 2-acetylnaphtho[2,3-b]furan-4,9-dione (BBI608)

A small scale DP3_19v1 formulation was prepared similarly as the DP3_19 in Example 2.

Large-scale DP3_19v1 formulations were prepared by using the components in Table 4.

TABLE 4

| Component | mg | wt-% |
|---|---|---|
| 2-acetylnaphtho[2,3-b]furan-4,9-dione | 80 | 16.66 |
| Vitamin E TPGS | 40 | 8.33 |
| Croscarmellose sodium | 80 | 16.66 |
| Kollidon VA 64 | 80 | 16.66 |
| Mannitol | 200 | 41.64 |
| Total weight | 480 | — |

### Example 4. Preparation of Pharmaceutical Tablets T-45 & T-46 of 2-acetylnaphtho[2,3-b]furan-4,9-dione (BBI608)

Small scale T-45 and T46 formulations were prepared similarly as the DP3_19 described in Example 2.

### Example 5. Comparison of Plasma Concentration of 2-acetylnaphtho[2,3-b]furan-4,9-dione (BBI608) Resulting from Administration of DP3_19 and DP3_19v1

For each formulation, an appropriate amount of the mixture was weighed to achieve 10 or 20 mg BBI608. 1 mL water was added to make suspensions for 100 mg/kg or 200 mg/kg, respectively, for the designated dose regimens.

Plasma samples were collected from the test subjects according to the experiment design. 5 μL of ACN or standard solutions of BBI608 (5, 10, 20 and 50 μg/mL BBI608) was added to 50 μL of blank plasma and the mixture was mixed briefly with vortex to prepare standard samples. Glass vials were used for the extraction and samples were prepared on ice.

5 μL of ACN was added to a test sample and the mixture was mixed briefly with vortex. 5 μL of internal standard solution (IS, 10 μg/mL) was added to both the standard and the test sample above and the mixture was mixed briefly with vortex. 150 μL of ACN containing 1% formic acid was added to the mixture to precipitate protein and dissociate BBI608 from protein in the plasma sample.

The sample was centrifuged at 13,000 rpm for 5 min at room temperature and 40 μL of supernatant was collected and injected into HPLC (HPLC Column: Phenomenex Luna C18(2), 5 μm, 250×4.6 mm; Mobile Phase: 10 mM Potassium Phosphate (pH 6.8):Acetonitrile (50:50); Flow Rate: 1.0 mL/min; Injection Volume: 40 μL; Column Temperature: Room temperature; Detector Wavelength: 254 nm).

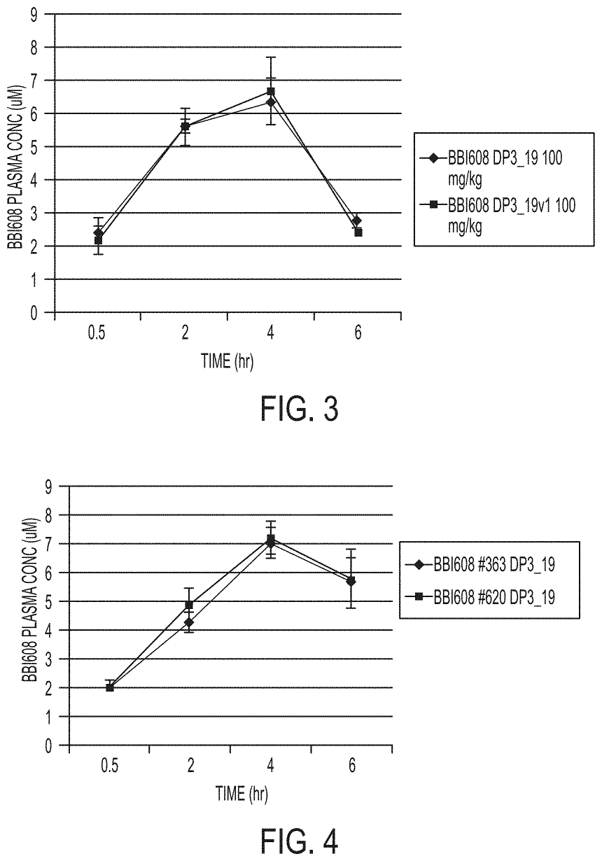

As shown in FIG. 3, DP3_19 and DP3_19v1 had substantially plasma concentrations from 0.5 to 6 hours after dosing.

### Example 6. Comparison of Plasma Concentration of 2-acetylnaphtho[2,3-b]furan-4,9-dione (BBI608) Resulting from Administration of DP3_19v1 with and without Sodium Lauryl Sulfate (SLS)

The experiment was conducted similarly to that in Example 5. As shown in FIG. 4, DP3_19 with or without sodium lauryl sulfate had substantially plasma concentrations from 0.5 to 6 hours after dosing.

### Example 7. Comparison of Plasma Concentration of 2-acetylnaphtho[2,3-b]furan-4,9-dione (BBI608) Resulting from Administration of DP2A and DP3_19v1

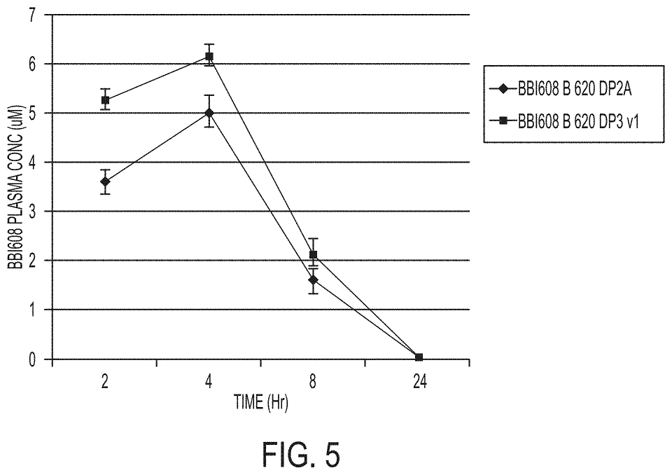

The experiment was conducted similarly to that in Example 5. As shown in FIG. 5, dosing of DP3_19v1 showed higher plasma concentrations throughout the testing period (from 2 to 24 hours after dosing) than dosing of DP2A.

### Example 8. Comparison of Plasma Concentration of 2-acetylnaphtho[2,3-b]furan-4,9-dione (BBI608) Resulting from Oral Administration of DP2A, DP3_19v1, T-45, and T-46

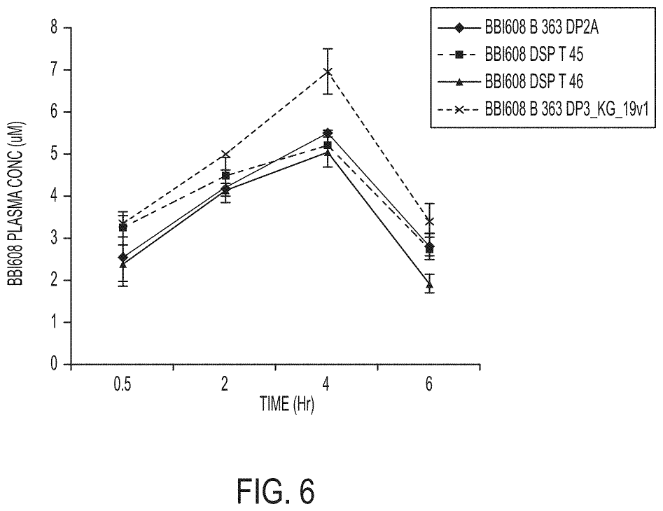

The experiment was conducted similarly to that in Example 5. As shown in FIG. 6, dosing of DP3_19v1 showed higher plasma concentrations in the testing period (from 0.5 to 6 hours after dosing) than dosing of DP2A, T-45, and T-46; dosing of T-45 showed a similar initial plasma concentration as dosing of DP3_19v1; dosing of T-46 and DP2A showed similar initial plasma concentration; and dosing of T-46 showed the lowest plasma concentration at 6 hours after dosing among all the tested formulations.

### Example 13. Metabolism Measurement of 2-acetylnaphtho[2,3-b]furan-4,9-dione in Human Liver Cytosol Fractions

A reaction solution obtained by dissolving a human liver cytosol (0.1 mg/mL) and NADPH (1 mmol/L) in a phosphate buffer solution was prepared. Into the reaction solution, [¹⁴C]-labeled labeled 2-acetylnaphtho[2,3-b]furan-4,9-dione was added such that the dione concentration is 0.5 μmol/L, and thereby the reaction was started. After incubation at 37° C. for a given time, 0.1% formic acid-containing acetonitrile was added to terminate the reaction, and the mixture was centrifuged. After the supernatant was evaporated to dryness under a stream nitrogen gas, re-dissolving solvent [10 mmol/L ammonium acetate:acetonitrile, 9:1 (v/v)] was used to re-dissolve it, and then Radio-HPLC was used to measure radioactivity.

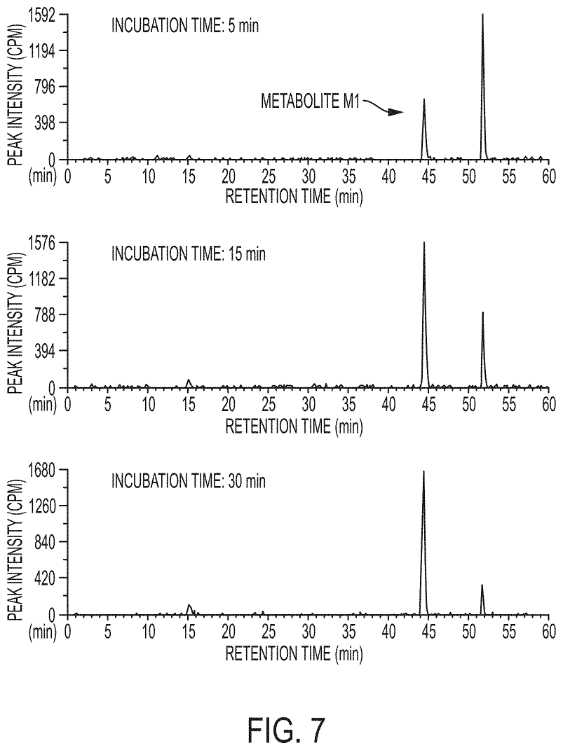

This experiment showed that the [¹⁴C]-labeled 2-acetylnaphtho[2,3-b]furan-4,9-dione was time-dependently metabolized in a liver cytosol to mainly produce a reduced-type metabolite, 2-(1-hydroxyethyl)naphtho[2,3-b]furan-4,9-dione (M1), was produced (FIG. 7).

### Example 14. Identification of a Metabolic Enzyme of 2-acetylnaphtho[2,3-b]furan-4,9-dione

To a reaction solution obtained by dissolving a human liver cytosol (0.1 mg/mL) and NADPH (1 mmol/L) in a phosphate buffer solution, quercitrin (10 μmol/L or 100 μmol/L), which is a CR inhibitor, and phenolphthalein or medroxyprogesterone 17-acetate (1.0 μmol/L or 10 μmol/L), which is an AKR inhibitor, were added. Further, [¹⁴C]-labeled 2-acetylnaphtho[2,3-b]furan-4,9-dione was added to the reaction solution such that the dione concentration is 0.5 μmol/L. After incubation at 37° C. for 5 minutes, Radio-HPLC was used to measure radioactivity.

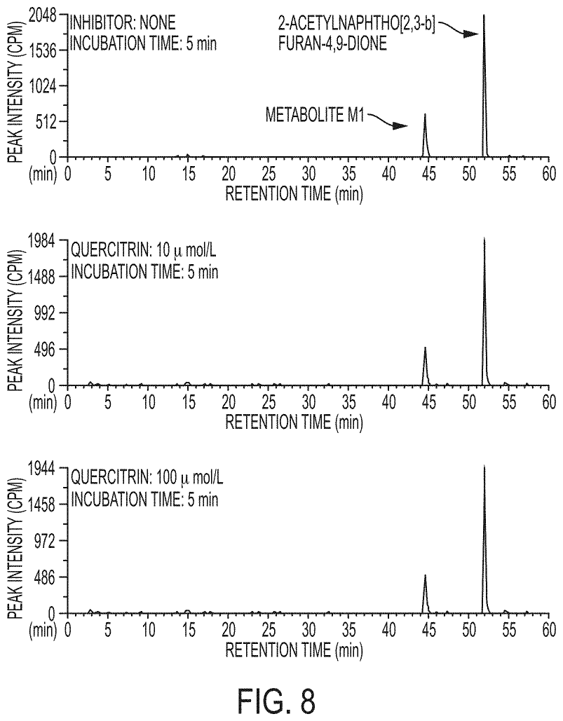

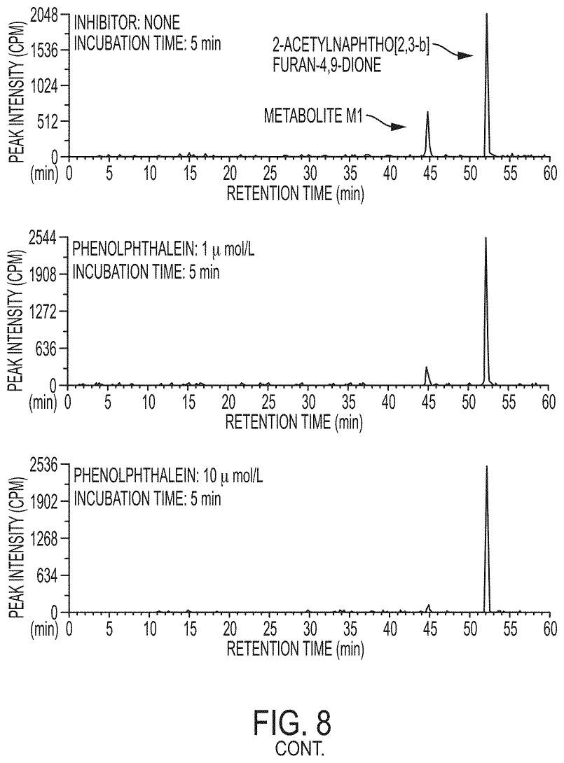

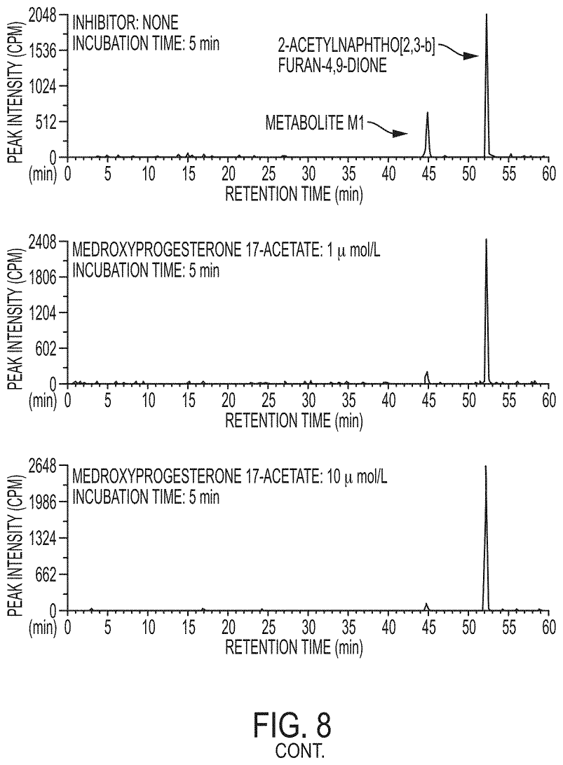

This experiment showed that the metabolism of 2-acetylnaphtho[2,3-b]furan-4,9-dione was weakly inhibited in the case of quercitrin only when the treatment at 100 μmol/L was performed, while in the case of phenolphthalein or medroxyprogesterone 17-acetate, the metabolism of 2-acetylnaphtho[2,3-b]furan-4,9-dione was more strongly inhibited than quercitrin (FIG. 8). A conclusion may be drawn that the AKR and CR were involved in the metabolism of 2-acetylnaphtho[2,3-b]furan-4,9-dione.

### Example 15. Screening for an Inhibitor of a Metabolic Enzyme

To a reaction solution obtained by dissolving a human liver cytosol (0.1 mg/mL) and NADPH (1 mmol/L) in a phosphate buffer solution, a test drug (10 μmol/L) was added. Then, 2-acetylnaphtho[2,3-b]furan-4,9-dione was added to be 0.5 μmol/L and thereby the reaction was started. After incubation at 37° C. for 5 minutes, 50 nmol/L phenytoin solution, which had been dissolved in 0.1% formic acid-containing acetonitrile, was added and the reaction was terminated. After centrifugation, the resulting supernatant was measured using LC-MS/MS to quantify the residual amount of 2-acetylnaphtho[2,3-b]furan-4,9-dione, and the inhibition intensity of the test drugs was evaluated by comparison with the control group.

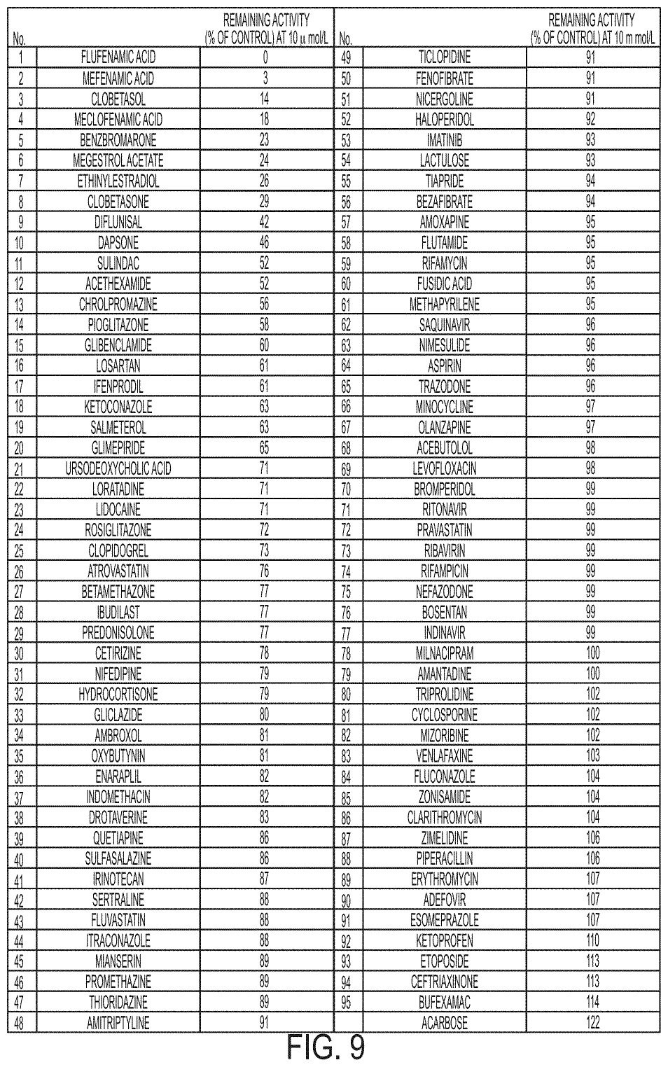

This experiment showed that the metabolism of 2-acetylnaphtho[2,3-b]furan-4,9-dione was inhibited by flufenamic acid, mefenamic acid, clobetasol, meclofenamic acid, benzbromarone, ethynyl estradiol, clobetasone, dapsone, sulindac, acetohexamide, chlorpromazine, pioglitazone, glibenclamide, losartan, ifenprodil, ketoconazole, salmeterol, and glimepiride (FIG. 9).

### Example 16. Identification of an AKR High-Expression Cell Line

Total RNAs respectively prepared from human cancer cell lines (A549, H460, HCT116, and HT29) were used to carry out DNA chip analysis. The DNA chip analysis was carried out using Gene Chip Human Genome HG133A and B (made by Affymetrix, Inc.).

Specifically, the analysis was carried out according to the following procedure: (1) preparation of cDNA from the total RNAs; (2) preparation of the labeled cRNA from the cDNA; (3) fragmentation of the labeled cRNA; (4) hybridization of the fragmented cRNA and a probe array; (5) dyeing the probe arrays; (6) scan of the probe arrays; and (7) gene expression analysis.

(1) Preparation of cDNA from the Total RNAs

An 11 μL mixture solution containing 100 μmol of T7-(dT)24 primer (made by Amersham) and 10 μg of each total RNA prepared from respective cancer cell lines was heated at 70° C. for 10 minutes and then cooled on ice. After cooling, 4 μL of 5× First Strand cDNA Buffer included in Superscript Choice System For Cdna Synthesis (made by Gibco-BRL), 2 μL of 0.1M DTT included in the kit, and 1 μL of 10 mM dNTP Mix included in the kit were added, and the mixture solution was heated at 42° C. for 2 minutes. Further, 2 μL (400U) of Super ScriptII RT included in the kit was added, then the mixture solution was heated at 42° C. for 1 hour and cooled on ice. After cooling, 91 μL of DEPC-treated water (made by Nacalai Tesque, Inc.), 30 μL of 5× Second Strand Reaction Buffer included in the kit, 3 μL of 10 mM dNTP Mix, 1 μL (10U) of E. coli DNA Ligase included in the kit, 4 μL (40U) of E. coli DNA Polymerase I included in the kit, and 1 μL (2U) of E. coli RNaseH included in the kit were added to allow the mixture solution to react at 16° C. for 5 minutes. Then, 2 μL (10U) T4 DNA Polymerase included in the kit was added. After reacting 16° C. for 5 minutes, 10 μL of 0.5M EDTA was added. Then, 162 μL of a phenol/chloroform/isoamyl alcohol solution (made by Nippon Gene Co., Ltd.) was added and mixed. The mixture solution was transferred into Phase Lock Gel Light (made by Eppendorf AG.), which had been previously centrifuged at room temperature at 14,000 rpm for 30 seconds. After centrifugation at room temperature at 14,000 rpm for 2 minutes, 145 μL of an aqueous layer was transferred to an Eppendorf tube. To the resulting solution, 72.5 μL of 7.5 M ammonium acetate solution and 362.5 μL of ethanol were added and mixed. The solution was then centrifuged at 4° C. at 14,000 rpm for 20 minutes. After centrifugation, the supernatant was discarded to obtain a DNA pellet including the cDNA made.

Then, 0.5 mL of 80% ethanol was added to the pellet. After centrifugation at 4° C. at 14,000 rpm for 5 minutes, the supernatant was discarded. After the same operation was performed again, the pellet was dried and dissolved in 12 μL of DEPC-treated water.

By the above operation, cDNAs were obtained from total RNAs respectively prepared from human cancer cell strains.

(2) Preparation of Labeled cRNA from the cDNA

Five microliters of each cDNA solution was mixed with 17 μL of DEPC-treated water, 4 μL of 10× HY Reaction Buffer included in BioArray High Yield RNA Transcript Labeling Kit (made by ENZO), 4 μL of 10× Biotin Labeled Ribonucleotides included in the kit, 4 μL of 10×DTT included in the kit, 4 μL of 10×RNase Inhibitor Mix included in the kit, and 2 μL of 20×T7 RNA Polymerase included in the kit to allow the mixture to react at 37° C. for 5 hours. After reaction, 60 μL of DEPC-treated water was added to the reaction solution, and then the prepared and labeled cRNA was purified using RNeasy Mini Kit in accordance with the attached protocol.

(3) Fragmentation of the Labeled cRNA

Forty microliters of a reaction solution obtained by adding 8 μL of 5× Fragmentation Buffer (200 mM tris-acetic acid pH 8.1 (made by Sigma), 500 mM potassium acetate (made by Sigma), and 150 mM magnesium acetate (made by Sigma)) to a solution containing 20 μg of each labeled cRNA, was heated at 94° C. for 35 minutes, and then was put on ice. By this, the labeled RNA was fragmented.

(4) Hybridization of the Fragmented cRNA and a Probe Array

Forty microliters of each fragmented cRNA was mixed with 4 μL of 5 nM Control Oligo B2 (made by Amersham), 4 μL of 100× Control cRNA Cocktail, 40 μg of Herring sperm DNA (made by Promega KK.), 200 μg of Acetylated BSA (made by Gibco-BRL), 200 μL of 2×MES Hybridization Buffer (200 mM MES, 2M [Na+], 40 mM EDTA, 0.02% Tween 20 (made by Pierce), pH 6.5-6.7), and 144 μL of DEPC-treated water to obtain 400 μL of a hybridization cocktail. The resulting hybridization cocktail was each heated at 99° C. for 5 minutes, and further heated at 45° C. for 5 minutes. After heating, it was centrifuged at room temperature at 14,000 rpm for 5 minutes to obtain the hybridization cocktail supernatant.

Meanwhile, Human genome HG133A and B probe arrays (made by Affymetrix, Inc.) filled with 1×MES hybridization buffer were rotated in a hybridization oven at 45° C. at 60 rpm for 10 minutes, and then the 1×MES hybridization buffer was removed to prepare probe arrays. Two hundred microliters of the hybridization cocktail supernatant obtained in the above was added to the probe arrays respectively, and they were rotated in a hybridization oven at 45° C. at 60 rpm for 16 hours to obtain probe arrays hybridized with the fragmented cRNA.

(5) Dyeing the Probe Arrays

The hybridization cocktail from each of the hybridized probe arrays obtained in the above was recovered and removed, and then was filled with Non-Stringent Wash Buffer (6×SSPE (dilution of 20×SSPE (made by Nacalai Tesque, Inc.)), 0.01% Tween 20, and 0.005% Antifoam0-30 (made by Sigma)). Then, the probe array hybridized with the fragmented cRNA was installed into the predetermined position of Gene Chip Fluidics Station 400 (made by Affymetrix, Inc.) in which Non-Stringent Wash Buffer and Stringent Wash Buffer (100 mM MES, 0.1M NaCl, and 0.01% Tween 20) are placed. Then, in accordance with a dyeing protocol EuKGE-WS2, dyeing was carried out using a primary solution (10 μg/mL Streptavidin Phycoerythrin (SAPE) (made by Molecular Probe), 2 mg/mL acetylated BSA, 100 mM MES, 1M NaCl (made by Ambion), 0.05% Tween 20, and 0.005% Antifoam 0-30) and a secondary dyeing solution (100 μg/mL Goat IgG (made by Sigma), 3 μg/mL Biotinylated Anti-Streptavidin antibody (made by Vector Laboratories), 2 mg/mL Acetylated BSA, 100 mM MES, 1M NaCl, 0.05% Tween 20, and 0.005% Antifoam 0-30).

(6) Scan of the Probe Arrays and (7) Gene Expression Analysis

Each of the dyed probe arrays was subjected to HP Gene Array Scanner (made by Affymetrix, Inc.) to read a dyeing pattern. Based on the dyed pattern, gene expression on the probe arrays was analyzed by Gene Chip Workstation System (made by Affymetrix, Inc.). Then, the normalization and the comparison analysis of gene expression were carried out in accordance with the analysis protocol.

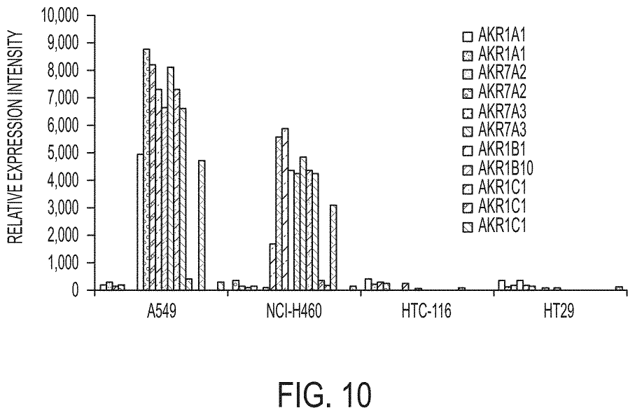

This experiment showed that AKR1B1, 1B10, 1C1, 1C2, and 1C3 were highly expressed in A549 and H460 (FIG. 10).

### Example 17. Increase of the Intracellular Concentration of 2-acetylnaphtho[2,3-b]furan-4,9-dione in a Cultured Cell by a Metabolic Inhibitor

A549 cells or H460 cells were seeded onto a 96-well culture plate (made by Corning Inc.) at 2×10⁴/well, and then cultured at 37° C. under 5% carbon dioxide atmosphere in an incubator for 48 hours. After which, they were cultured for 2 hours with 2-acetylnaphtho[2,3-b]furan-4,9-dione in a final concentration of 1.0 μmol/L and in the presence or absence of 30 μmol/L or 100 μmol/L of mefenamic acid or flufenamic acid, and then washed with PBS. After that, 80% methanol was added to each well to obtain a cell lysate. The cell lysate was analyzed using LC-MS/MS to determine the intracellular concentration of 2-acetylnaphtho[2,3-b]furan-4,9-dione.

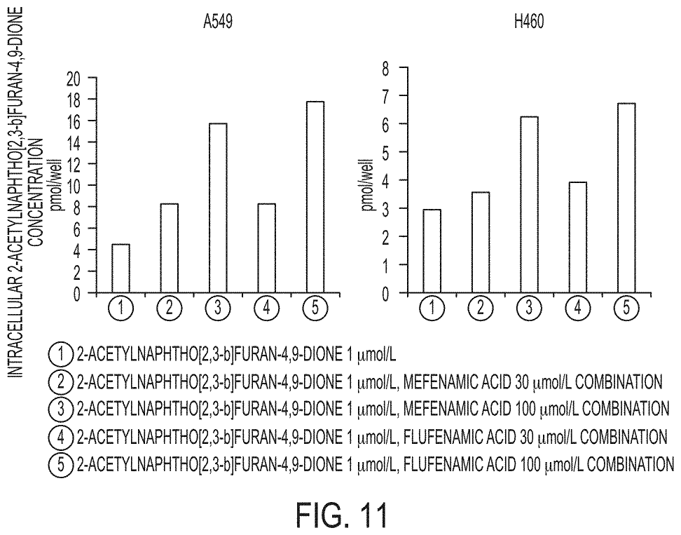

This experiment showed that the intracellular concentration of 2-acetylnaphtho[2,3-b]furan-4,9-dione in the presence of mefenamic acid or flufenamic acid increased (FIG. 11).

### Example 18. Improvement in Cytotoxic Effect of 2-acetylnaphtho[2,3-b]furan-4,9-dione in a Culture Cell by a Metabolic Inhibitor

A549 cells or H460 cells were seeded onto a 96-well culture plate (Corning) at 2×10⁴/well, and then cultured at 37° C. under 5% carbon dioxide atmosphere in an incubator for 48 hours. 2-Acetylnaphtho[2,3-b]furan-4,9-dione was added to each well such that the final concentration is 1.0 μmol/L. After which, they were cultured in the presence or absence of a test compound (30 μmol/L of flufenamic acid, 100 μmol/L of flufenamic acid, or 100 μmol/L of mefenamic acid as a final concentration) for 6 hours, Prest Blue (Life Technologies) was added, and then incubated for 1 to 2 hours. After the incubation, absorbance at 570 nm was measured to evaluate the cytotoxicity. Absorbance of a well to which a cell had not been added (Ablank) is defined as the background, the cell survival rate was calculated using the following formula to divide a value obtained by multiplying 100 by a value obtained by subtracting the background absorbance from the absorbance of each well (Asample), by a value obtained by subtracting Ablank from the value (Acontrol) of a well in which a cell with no treatment with 2-acetylnaphtho[2,3-b]furan-4,9-dione or a metabolic inhibitor was contained.

Cell survival rate (%)=(Asample−Ablank)×100/(Acontrol−Ablank)

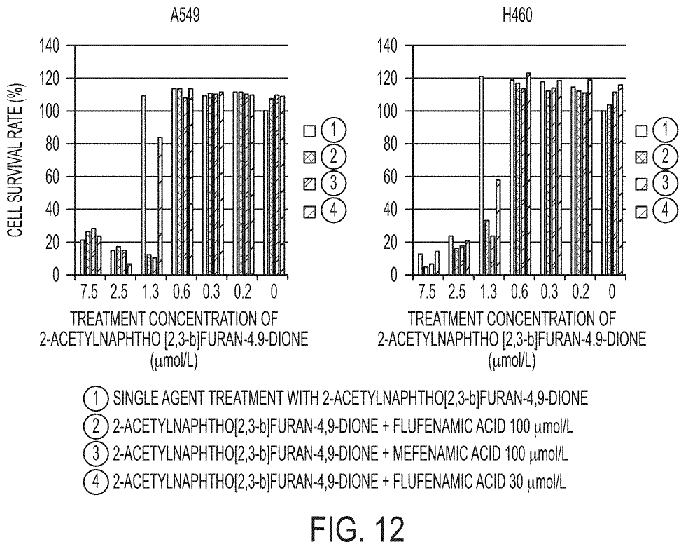

From the result of the evaluation, in both cells of A549 cell and H460 cell, in the single agent treatment with 1.3 μmol/L of 2-acetylnaphtho[2,3-b]furan-4,9-dione, the cell survival rate was 100% or more, while addition of 100 μmol/L of flufenamic acid or mefenamic acid significantly decreased the cell survival rate. It was found that the combination with flufenamic acid or mefenamic acid enhanced the cytotoxically active effect of 2-acetylnaphtho[2,3-b]furan-4,9-dione (FIG. 12).

### Example 19. Concentration change of 2-acetylnaphtho[2,3-b]furan-4,9-dione in Plasma and Tumor by Combination with a Metabolic Inhibitor in Tumor-Bearing Mice

The following administration solution (1), (2) or (3) was orally administered to 7 week aged mice (BALB/cAnNCrlCrlj, female, Charles River Laboratories Japan, Inc.) in a single time.

(1) 100 mg/kg of 2-acetylnaphtho[2,3-b]furan-4,9-dione

(2) 100 mg/kg of 2-acetylnaphtho[2,3-b]furan-4,9-dione and 100 mg/kg of mefenamic acid

(3) 100 mg/kg of 2-acetylnaphtho[2,3-b]furan-4,9-dione and 300 mg/kg of mefenamic acid

The blood was collected 2, 6, and 16 hours after the administration, and the blood was centrifuged to give plasma. Methanol was added to the plasma such that the final concentration of methanol is 80%, and then it was centrifuged. Further, protein-removing treatment was carried out by filtering through a filter, and then 2-acetylnaphtho[2,3-b]furan-4,9-dione was detected and quantified using LC-MS/MS (API4000, AB SCIEX). Meanwhile, a tumor mass was collected at the same time as the blood collection, 2-acetylnaphtho[2,3-b]furan-4,9-dione in the tumor was detected and quantified.

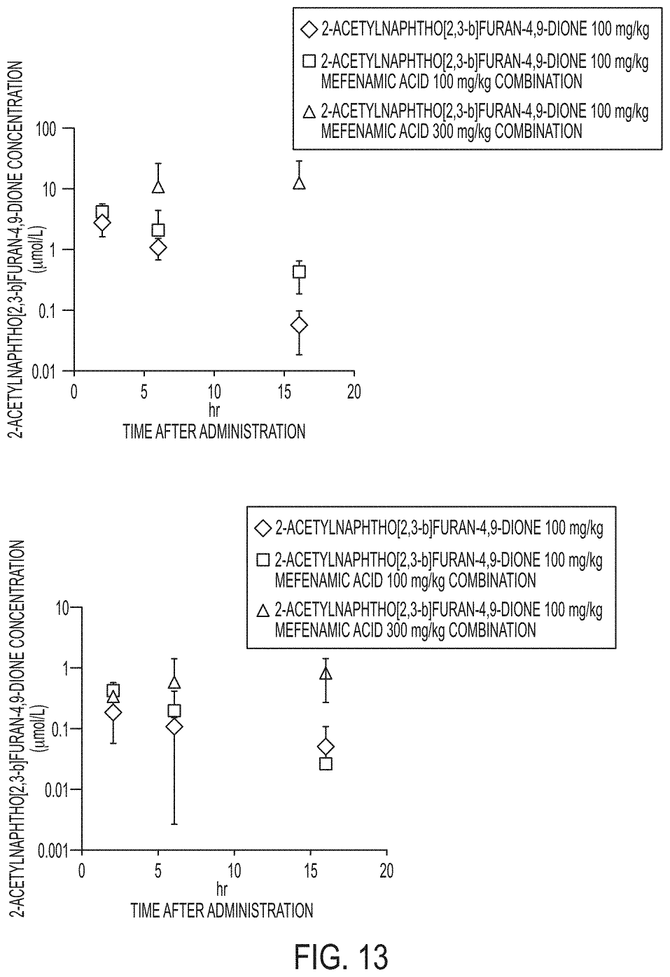

From the result of the evaluation, it was found that in comparison with the group of single agent administration of 2-acetylnaphtho[2,3-b]furan-4,9-dione, the concentration of 2-acetylnaphtho[2,3-b]furan-4,9-dione in plasma was higher in the group of combination with mefenamic acid (FIG. 13). Moreover, in the concentration in tumor, the concentration of 2-acetylnaphtho[2,3-b]furan-4,9-dione was higher in the group of combination of 2-acetylnaphtho[2,3-b]furan-4,9-dione with 300 mg/kg of mefenamic acid than the single agent group of 2-acetylnaphtho[2,3-b]furan-4,9-dione (FIG. 13).

### Example 20. Concentration Change of 2-acetylnaphtho[2,3-b]furan-4,9-dione in Plasma by Combination with a Metabolic Inhibitor in Cancer-Bearing Mice

The following administration solution (1) or (2) was orally administered to 8 week aged mice (BALB/cAnNCrlCrlj, female, Charles River Laboratories Japan, Inc.) in a single time.

(1) 100 mg/kg of 2-acetylnaphtho[2,3-b]furan-4,9-dione

(2) 100 mg/kg of 2-acetylnaphtho[2,3-b]furan-4,9-dione and 25 mg/kg of sulindac

Two hours after the administration, the blood was collected and then centrifuged to give plasma. Methanol was added to the plasma such that the final concentration of methanol is 80%, and then it was centrifuged. Further, protein-removing treatment was carried out by filtering through a filter, and then 2-acetylnaphtho[2,3-b]furan-4,9-dione was detected and quantified using LC-MS/MS (API4000, AB SCIEX).

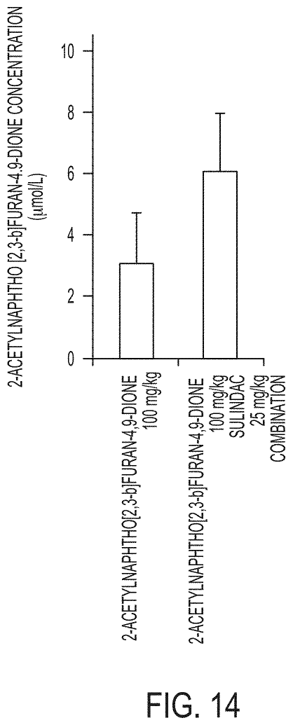

This experiment showed that in comparison with the group of single agent administration 2-acetylnaphtho[2,3-b]furan-4,9-dione, the concentration of 2-acetylnaphtho[2,3-b]furan-4,9-dione in plasma was higher in the group of combination with sulindac (FIG. 14).

### Example 21. Anti-Tumor Effect by a Combination 2-acetylnaphtho[2,3-b]furan-4,9-dione with Mefenamic Acid in Tumor-Bearing Mice

A549 cells of 5×10⁶ per individual were transplanted to a right belly part of a 5 week aged mouse (BALB/cAnNCrlCrlj, male, Charles River Laboratories Japan, Inc.). After one week, the following administration solution (1), (2), (3), (4) or (5) was orally administered in a single time.

(1) 100 mg/kg of mefenamic acid

(2) 30 mg/kg of 2-acetylnaphtho[2,3-b]furan-4,9-dione

(3) 100 mg/kg of 2-acetylnaphtho[2,3-b]furan-4,9-dione

(4) 30 mg/kg of 2-acetylnaphtho[2,3-b]furan-4,9-dione and 100 mg/kg of mefenamic acid

(5) 100 mg/kg of 2-acetylnaphtho[2,3-b]furan-4,9-dione and 100 mg/kg of mefenamic acid

According to a schedule in which an administration solution or an administration solution without an agent was continuously administered for five days and then medication was ceased for two days and continuous administration for five days was carried out again, it was orally administered one time a day. The major axis and the minor axis of a tumor mass were measured by vernier calipers at 2 to 5 day intervals, and the tumor volume was calculated by substituting them into the formula: (minor axis)²×(major axis)/2.

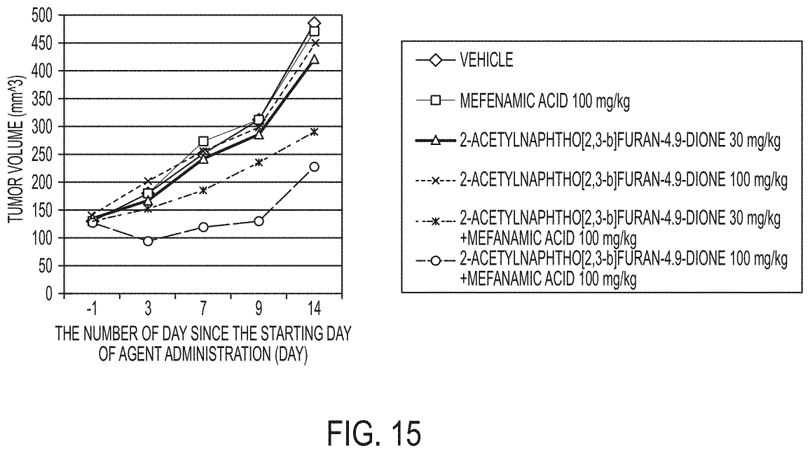

Although mefenamic acid as a single agent has no anti-tumor activity, the use of 2-acetylnaphtho[2,3-b]furan-4,9-dione and mefenamic acid in combination significantly improved the anti-tumor activity in comparison with 2-acetylnaphtho[2,3-b]furan-4,9-dione as a single agent (FIG. 15).

Without being limited to any particular theory, the combinations of the present disclosure seem to improve the pharmacokinetics and pharmacodynamics of 2-acetylnaphtho[2,3-b]furan-4,9-dione and enhance its anti-tumor effect and, accordingly, lower dosages or fewer administration of 2-acetylnaphtho[2,3-b]furan-4,9-dione may be necessary to prevent and/or treat cancer.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described; accordingly, all suitable modifications and equivalents may be resorted to, falling within the scope of the present disclosure.

Moreover, those of ordinary skill in the art will appreciate that the conception upon which this disclosure is based may readily be used as a basis for designing other pharmaceutical compositions and pharmaceutical tablets for carrying out the several purposes of the present disclosure. Accordingly, the claims are not intended to be limited by the foregoing description.

1. A pharmaceutical composition comprising:
at least one compound selected from compounds having formula (I) 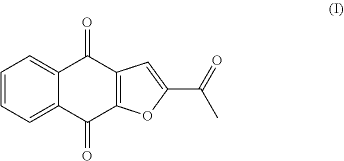 [SMILES: CC(=O)c1cc2c(o1)C(=O)c1ccccc1C2=O]
prodrugs thereof, pharmaceutically acceptable salts of any of the foregoing, and
pharmaceutically acceptable solvates of any of the foregoing;
at least one binder;
at least one disintegrant;
at least one other excipient; and
at least one component chosen from at least one lubricant and at least one surfactant.

2-3. (canceled)

4. The pharmaceutical composition claim 1, wherein
the at least one binder is Kollidon VA 64 (copovidone/vinylpyrrolidone-vinyl acetate copolymer);
the at least one disintegrant is croscarmellose sodium;
the at least one pharmaceutical excipient is mannitol; and
the at least one surfactant is Vitamin E TPGS (D-α-tocopheryl polyethylene glycol 1000 succinate).

5. The pharmaceutical composition of claim 4, wherein:
the at least one compound is in an amount ranging from about 5 wt-% to about 25 wt-%;
the Kollidon VA 64 (copovidone/vinylpyrrolidone-vinyl acetate copolymer) is in an amount ranging from about 5 wt-% to about 25 wt-%;
the croscarmellose sodium is in an amount ranging from about 5 wt-% to about 25 wt-%;
the mannitol is in an amount ranging from about 20 wt-% to about 60 wt-%; and
the Vitamin E TPGS (D-α-tocopheryl polyethylene glycol 1000 succinate) is in an amount ranging from about 1 wt-% to about 20 wt-%.

6. (canceled)

7. The pharmaceutical composition of claim 5, wherein:
the at least one compound is in an amount of about 16.7 wt-%;
the Kollidon VA 64 (copovidone/vinylpyrrolidone-vinyl acetate copolymer) is in an amount of about 16.7 wt-%;
the croscarmellose sodium is in an amount of about 16.7 wt-%;
the mannitol is in an amount of about 41.67 wt-%; and
the Vitamin E TPGS (D-α-tocopheryl polyethylene glycol 1000 succinate) is in an amount of about 8.33 wt-%.

8. The pharmaceutical composition of claim 4, wherein:
the at least one compound is in an amount ranging from about 30 mg to about 130 mg;
the Kollidon VA 64 (copovidone/vinylpyrrolidone-vinyl acetate copolymer) is in an amount ranging from about 30 mg to about 130 mg;
the croscarmellose sodium is in an amount ranging from about 30 mg to about 130 mg;
the mannitol is in an amount ranging from about 100 mg to about 300 mg; and
the Vitamin E TPGS (D-α-tocopheryl polyethylene glycol 1000 succinate) is in an amount ranging from about 5 mg to about 75 mg.

9. (canceled)

10. The pharmaceutical composition of claim 8, wherein:
the at least one compound is in an amount of about 80 mg;
the Kollidon VA 64 (copovidone/vinylpyrrolidone-vinyl acetate copolymer) is in an amount of about 80 mg;
the croscarmellose sodium is in an amount of about 80 mg;
the mannitol is in an amount of about 200 mg; and
the Vitamin E TPGS (D-α-tocopheryl polyethylene glycol 1000 succinate) is in an amount of about 40 mg.

11. The pharmaceutical composition of claim 4, wherein:
the at least one compound is in an amount ranging from about 5 wt-% to about 25 wt-%;
the Kollidon VA 64 (copovidone/vinylpyrrolidone-vinyl acetate copolymer) is in an amount ranging from about 5 wt-% to about 25 wt-%;
the croscarmellose sodium is in an amount ranging from about 15 wt-% to about 55 wt-%;
the mannitol is in an amount ranging from about 5 wt-% to about 25 wt-%; and
the Vitamin E TPGS (D-α-tocopheryl polyethylene glycol 1000 succinate) is in an amount ranging from about 5 wt-% to about 25 wt-%.

12. (canceled)

13. The pharmaceutical composition of claim 11, wherein:
the at least one compound is in an amount of about 16.7 wt-%;
the Kollidon VA 64 (copovidone/vinylpyrrolidone-vinyl acetate copolymer) is in an amount of about 16.7 wt-%;
the croscarmellose sodium is in an amount of about 33.33 wt-%;
the mannitol is in an amount of about 16.7 wt-%; and
the Vitamin E TPGS (D-α-tocopheryl polyethylene glycol 1000 succinate) is in an amount of about 16.7 wt-%.

14. The pharmaceutical composition of claim 4, wherein:
the at least one compound is in an amount ranging from about 30 mg to about 130 mg;
the Kollidon VA 64 (copovidone/vinylpyrrolidone-vinyl acetate copolymer) is in an amount ranging from about 30 mg to about 130 mg;
the croscarmellose sodium is in amount ranging from about 50 mg to about 250 mg;
the mannitol is in an amount ranging from about 30 mg to about 130 mg; and
the Vitamin E TPGS (D-α-tocopheryl polyethylene glycol 1000 succinate) is in an amount ranging from about 30 mg to about 130 mg.

15. (canceled)

16. The pharmaceutical composition of claim 14, wherein:
the at least one compound is in an amount of about 80 mg;
the Kollidon VA 64 (copovidone/vinylpyrrolidone-vinyl acetate copolymer) is in an amount of about 80 mg;
the croscarmellose sodium is in an amount of about 160 mg;
the mannitol is in an amount of about 80 mg; and
the Vitamin E TPGS (D-α-tocopheryl polyethylene glycol 1000 succinate) is in an amount of about 80 mg.

17-18. (canceled)

19. The pharmaceutical composition of claim 1, wherein:
the at least one binder is partially hydrolyzed polyvinyl alcohol;
the at least one disintegrant is selected from sodium carboxymethyl starch and low substituted hydroxypropylcellulose;
the at least one filler is microcrystalline cellulose; and
the at least one lubricant is magnesium stearate.

20. The pharmaceutical composition of claim 19, wherein:
the at least one compound is in an amount ranging from about 25 wt-% to about 75 wt-%;
the partially hydrolyzed polyvinyl alcohol is in an amount ranging from about 0.5 wt-% to about 5 wt-%;
the low substituted hydroxypropylcellulose is in an amount ranging from about 5 wt-% to about 25 wt-%;
the microcrystalline cellulose is in an amount ranging from about 15 wt-% to about 45 wt-%; and
the magnesium stearate is in an amount ranging from about 0.1 wt-% to about 2 wt-%.

21. (canceled)

22. The pharmaceutical composition of claim 20, wherein:
the at least one compound is in an amount of about 50.0 wt-%;
the partially hydrolyzed polyvinyl alcohol is in an amount of about 3.0 wt-%;
the low substituted hydroxypropylcellulose is in an amount of about 15.0 wt-%;
the microcrystalline cellulose is in an amount of about 31.0 wt-%; and
the magnesium stearate is in an amount of about 1.0 wt-%.

23. The pharmaceutical composition of claim 19, wherein:
the at least one compound is in an amount ranging from about 30 mg to about 130 mg;
the partially hydrolyzed polyvinyl alcohol is in an amount ranging from about 3 mg to about 6 mg;
the low substituted hydroxypropylcellulose is in an amount ranging from about 16 mg to about 32 mg;
the microcrystalline cellulose is in an amount ranging from about 35 mg to about 65 mg; and
the magnesium stearate is in an amount ranging from about 0.5 mg to about 2 mg.

24. (canceled)

25. The pharmaceutical composition of claim 23, wherein:
the at least one compound is in an amount of about 80.0 mg;
the partially hydrolyzed polyvinyl alcohol is in an amount of about 4.8 mg;
the low substituted hydroxypropylcellulose is in an amount of about 24.0 mg;
the microcrystalline cellulose is in an amount of about 49.6 mg; and
the magnesium stearate is in an amount of about 1.6 mg.

26. The pharmaceutical composition of claim 1, further comprising sodium lauryl sulfate in an amount ranging from about 0.1 wt-% to about 2 wt-% relative to the amount of about the compound.

27. The pharmaceutical composition of claim 1, wherein:
the at least one compound is in an amount ranging from about 20 wt-% to about 80 wt-%;
the sodium lauryl sulfate is in an amount ranging from about 0.1 wt-% to about 2 wt-%;
the partially hydrolyzed polyvinyl alcohol is in an amount ranging from about 0.5 wt-% to about 3 wt-%;
the low substituted hydroxypropylcellulose is in an amount ranging from about 5 wt-% to about 25 wt-%;
the sodium carboxymethyl starch is in an amount ranging from about 1 wt-% to about 7 wt-%;
the microcrystalline cellulose is in an amount ranging from about 15 wt-% to about 40 wt-%; and
the magnesium stearate is in an amount ranging from about 0.1 wt-% to about 2 wt-%.

28. (canceled)

29. The pharmaceutical composition of claim 1, wherein:
the at least one compound is in an amount of about 50.0 wt-%;
the sodium lauryl sulfate is in an amount of about 0.5 wt-%;
the partially hydrolyzed polyvinyl alcohol is in an amount of about 2.0 wt-%;
the low substituted hydroxypropylcellulose is in an amount of about 15.0 wt-%;
the sodium carboxymethyl starch is in an amount of about 4.0 wt-%;
the microcrystalline cellulose is in an amount of about 27.5 wt-%; and
the magnesium stearate is in an amount of about 1.0 wt-%.

30. The pharmaceutical composition of claim 1, wherein:
the at least one compound is in an amount ranging from about 30 mg to about 130 mg;
the sodium lauryl sulfate is in an amount ranging from about 0.1 mg to about 2 mg;
the partially hydrolyzed polyvinyl alcohol is in an amount ranging from about 1 mg to about 5 mg;
the low substituted hydroxypropylcellulose is in an amount ranging from about 10 mg to about 40 mg;
the sodium carboxymethyl starch is in an amount ranging from about 4 mg to about 9 mg;
the microcrystalline cellulose is in an amount ranging from about 30 mg to about 60 mg; and
the magnesium stearate is in an amount ranging from about 0.5 mg to about 2 mg.

31. (canceled)

32. The pharmaceutical composition of claim 30, wherein:
the at least one compound is in an amount of about 80.0 mg;
the sodium lauryl sulfate is in an amount of about 0.8 mg;
the partially hydrolyzed polyvinyl alcohol is in an amount of about 3.2 mg;
the low substituted hydroxypropylcellulose is in an amount of about 24.0 mg;
the sodium carboxymethyl starch is in an amount of about 6.4 mg;
the microcrystalline cellulose is in an amount of about 44.0 mg; and
the magnesium stearate is in an amount of about 1.6 mg.

33. (canceled)

34. A combination for treating and/or preventing cancer comprising:
(a) a first agent comprising 2-acetylnaphtho[2,3-b]furan-4,9-dione, a prodrug thereof, a pharmaceutically acceptable salt of any of the foregoing, or a pharmaceutically acceptable solvate any of the foregoing; and
(b) at least one second agent, wherein the at least one second agent is a metabolic inhibitor, a transporter inhibitor, and a combination thereof.

35-50. (canceled)

51. A method of treating cancer, comprising administering to a subject in need thereof, the pharmaceutical composition of claim 1.

52-74. (canceled)
